SEQUENCE LISTING (12 sequences)

SEQ ID NO: 1 - DNA, 23 bp, synthetic construct
  misc_feature 1..23: note = Forward primer for RPL19
  source 1..23: mol_type = other DNA
gaaggtcaaagggaatgtgttca

SEQ ID NO: 2 - DNA, 21 bp, synthetic construct
  misc_feature 1..21: note = Reverse primer for RPL19
  source 1..21: mol_type = other DNA
ccttgtctgccttcagcttgt

SEQ ID NO: 3 - DNA, 20 bp, synthetic construct
  misc_feature 1..20: note = Forward primer for D2R
  source 1..20: mol_type = other DNA
ccaagaacgtgagggctaag

SEQ ID NO: 4 - DNA, 20 bp, synthetic construct
  misc_feature 1..20: note = Reverse primer for D2R
  source 1..20: mol_type = other DNA
tgaggatgcgaaaggagaag

SEQ ID NO: 5 - DNA, 19 bp, synthetic construct
  misc_feature 1..19: note = Forward primer for D1R
  source 1..19: mol_type = other DNA
gagccaacctgaagacacc

SEQ ID NO: 6 - DNA, 21 bp, synthetic construct
  misc_feature 1..21: note = Reverse primer for D1R
  source 1..21: mol_type = other DNA
tgacagcatctccatttccag

SEQ ID NO: 7 - DNA, 21 bp, synthetic construct
  misc_feature 1..21: note = Forward primer for TH
  source 1..21: mol_type = other DNA
gccaaggacaagctcaggaac

SEQ ID NO: 8 - DNA, 20 bp, synthetic construct
  misc_feature 1..20: note = Reverse primer for TH
  source 1..20: mol_type = other DNA
atcaatggccagggtgtacg

SEQ ID NO: 9 - DNA, 21 bp, synthetic construct
  misc_feature 1..21: note = Forward primer for DAT
  source 1..21: mol_type = other DNA
aaatgctccgtgggaccaatg

SEQ ID NO: 10 - DNA, 20 bp, synthetic construct
  misc_feature 1..20: note = Reverse primer for DAT
  source 1..20: mol_type = other DNA
gtctcccgctcttgaacctc

SEQ ID NO: 11 - DNA, 20 bp, synthetic construct
  misc_feature 1..20: note = universal eubacterial primer 27Fmod
  variation 3: note = r at position 3 is g or a
  variation 12: note = m at position 12 is a or c
  source 1..20: mol_type = other DNA
agrgtttgatcmtggctcag

SEQ ID NO: 12 - DNA, 18 bp, synthetic construct
  misc_feature 1..18: note = universal eubacterial primer 519Rmodbio
  variation 3: note = n at position 3 is any nucleotide (a or g or c or t)
  variation 14: note = k at position 14 is g or t
  source 1..18: mol_type = other DNA
  variation 8: note = n at position 8 is any nucleotide (a or g or c or t)
gtnttacngcggckgctg

(19) United States
(12) Patent Application Publication
(10) Pub. No.: US 2024/0415895 A1
(43) Pub. Date: Dec. 19, 2024

(54) PREVENTION AND/OR TREATMENT OF REWARD DYSREGULATION DISORDERS

(71) Applicant: UNIVERSITÉ CATHOLIQUE DE LOUVAIN, Louvain-la-Neuve (BE)

(72) Inventors: Amandine EVERARD, Dion Valmont (BE); Alice DE WOUTERS D'OPLINTER, Bruxelles (BE); Geoffroy Olivier Ludovic MALLARET, Clermont-Ferrand (FR); Patrice CANI, Bruxelles (BE)

(73) Assignee: UNIVERSITÉ CATHOLIQUE DE LOUVAIN, Louvain-la-Neuve (BE)

(21) Appl. No.: 18/580,828

(22) PCT Filed: Jul. 20, 2022
(86) PCT No.: PCT/EP2022/070430

§ 371 Date: Jan. 19, 2024

(30) Foreign Application Priority Data
Jul. 20, 2021 (WO) PCT/EP2021/070303
Feb. 22, 2022 (EP) 22158054.1

(51) Int. Cl. A61K 35/74; A61P 3/04; A61P 25/00; A61P 25/30

(52) U.S. Cl. CPC A61K 35/74; A61P 3/04; A61P 25/00; A61P 25/30

(57) ABSTRACT

A composition including one or more bacteria selected from the genus Parabacteroides and/or an extract thereof and/or metabolites thereof. Also, the use of this composition in methods of preventing and/or treating reward dysregulation disorders, including mental disorders, neurological disorders, and combinations thereof, by administering this composition to an individual in need thereof.

### FIELD OF INVENTION

The present invention relates to the field of disorders related to reward dysregulation. In particular, the invention relates to compositions comprising one or more bacteria from the genus Parabacteroides and/or extracts and/or metabolites thereof for use in preventing and/or treating reward dysregulation disorders.

### BACKGROUND OF INVENTION

The reward system is often defined as being related to the aggregate of neural circuits that process appetitive stimuli, within the limbic system, the basal ganglia, the prefrontal cortex, the ventral tegmental area, and substantia nigra.

When the reward system is functioning properly, the anticipation or acquisition of a reward will catalyze a cascade of events involving neurotransmitters such as, e.g., dopamine, GABA, glutamate, serotonin, and norepinephrine.

Dysfunction in reward mechanisms can occur naturally (e.g., when dopamine levels decline upon social isolation, or when serotonin levels decline because of aging), or artificially (e.g., upon consumption of dopamine antagonist). Reward dysfunction may also occur upon illness or genetic disorders. Dysfunction in these mechanisms is characterized by reward learning and motivation deficits and emotional abnormalities, such as, e.g., a lack of pleasure or satisfaction, reduction in motivation, and emotional numbing.

For example, in the context of obesity, wherein overeating and consumption of calorie-dense food are major aspects contributing to a positive energy balance (energy input is greater than energy output) and the storage of fat, the reward system, that drives eating behaviors associated with pleasure, becomes the major driver for food intake. Palatable food, rich in fat and sugar, can stimulate dopaminergic neurons and induce a release of dopamine mainly in the cortico-limbic areas of the brain (including the striatum, nucleus accumbens and prefrontal cortex). However, obesity, which is often the result of long-term overeating, is associated with a reduction of dopamine concentration in response to palatable food intake and a downregulation of dopaminergic markers. The expressions of dopamine receptors 1 (D1R) and 2 (D2R) are decreased, as well as the rate-limiting synthetizing enzyme (tyrosine hydroxylase, TH) whereas the dopamine transporter (DAT) is increased. This altered functioning of the dopamine pathway has been suggested to feed the vicious circle of weight gain since it leads to an increase of the meal size of fatty and sweet food in an attempt to feel the same rewarding effect as before the development of obesity.

A reward dysregulation mechanism may also occur in many diseases including addiction-related disorder, affective disorders, obsessive compulsive disorders, schizophrenia, attention deficit hyperactivity disorders (ADHD), autism spectrum disorder, major depressive disorder (MDD), anxiety disorder and Parkinson's disease.

So far, therapy for reward dysregulation disorders may account for neuropharmacological compounds and/or psychotherapy.

There is therefore a need to provide the state of the art with alternative therapy to treat reward dysregulation disorders. In particular, there is a need to provide efficient therapy for reward dysregulation disorders.

### SUMMARY

The present invention relates to a composition comprising one or more bacteria from the genus Parabacteroides and/or an extract thereof and/or metabolites thereof, for use in preventing and/or treating reward dysregulation disorders.

In one embodiment, the bacteria from the genus Parabacteroides are selected in the group comprising or consisting of P. distasonis, P. goldsteinii, P. merdae, P. acidifaciens, P. bouchesdurhonensis, P. chartae, P. chinchilla, P. chongii, P. faecis, P. gordonii, P. johnsonii, P. massiliensis, P. pacaensis, P. provencensis, P. timonensis, Parabacteroides spp. and combinations thereof.

In one embodiment, the reward dysregulation disorder is selected in a group comprising or consisting of mental disorders, neurological disorders, and combinations thereof. In one embodiment, the mental disorder is selected in a group comprising or consisting of addiction-related disorder, eating-related disorder, affective disorders, obsessive compulsive disorders, schizophrenia, attention deficit hyperactivity disorders (ADHD), autism spectrum disorder, major depressive disorder (MDD), anxiety disorder, and the like. In one embodiment, the eating-related disorder is selected in a group comprising or consisting of anorexia, bulimia, overweight-related disorders, obesity-related disorders, and the like. In one embodiment, the addiction-related disorder is selected in a group comprising or consisting of alcohol-related addiction, drug-related addiction, game-related addiction, and the like. In one embodiment, the neurological disorder is selected in a group comprising or consisting of Parkinson's disease, Tourette Syndrome, and the like.

In one embodiment, the composition is to be administered to an animal individual, preferably a mammalian individual, more preferably a human individual.

In one embodiment, the composition is to be administered orally or rectally.

In one embodiment, the bacteria are to be administered at a dose comprised from about 1×10² CFU/g to about 1×10¹² CFU/g of the composition.

In one embodiment, the composition further comprises one or more beneficial microbe(s). In one embodiment, the one or more beneficial microbe(s) is/are selected in a group comprising or consisting of bacteria from the family Clostridiaceae, from the family Peptostreptococcaceae, from the family Prevotellaceae, from the family Methylobacteriaceae, from the genus Turicibacter, from the genus Coprococcus, from the genus Knoellia, from the genus Prevotella, from the genus Staphylococcus, from the genus Akkermansiaceae, and the like.

In one embodiment, the composition is in the form of a pharmaceutical composition further comprising a pharmaceutically acceptable carrier. In another embodiment, the composition is in the form of a nutritional composition further comprising a nutritionally acceptable carrier.

In one embodiment, the composition is comprised in a kit, which further comprises means to administer said composition.

### Definitions

In the present invention, the following terms have the following meanings:
“About”, when preceding a value, encompasses plus or minus 10%, or less, of the value of said figure. It is to be understood that the value to which the term “about” refers to is itself also specifically, and preferably, disclosed.
“Comprise” is intended to mean “contain”, “encompass” and “include”. In some embodiments, the term “comprise” also encompasses the term “consist of”.
“Bacteria from the genus Parabacteroides” refers to Gram-negative, obligatory anaerobic, non-spore forming, non-motile bacteria, which are able to grow on a culture medium containing 20% (w/v) bile. Bacteria belonging to the genus Parabacteroides may be easily identified by routine procedures, including physiological and biochemical approaches, assessment of their cellular fatty acid profiles, menaquinone profiles and their phylogenetic position, based on 16S rRNA gene sequence analysis.
“Isolated bacteria” refers to bacteria that are no longer in their natural and/or physiological biotope or habitat. For example, bacteria of interest from a microbiota may be collected and separated from other bacteria and further formulated within a composition. Bacterial separation may be performed according to standard protocols in the field of microbiology, such as, e.g., Gram coloration, antibiotic resistance, ability to grow on specific substrates/culture media, and protocols adapted therefrom.
“Enriched composition” refers to a composition in which the population density of bacteria from the genus Parabacteroides is enhanced within the total microbial population of the composition.
“Extract” refers to any fraction obtained from the bacteria of interest, or from culture media in which the bacteria of interest were cultured. In practice, extracts include cellular and extracellular extracts. In one embodiment, extracts according to the present invention include metabolites from the bacteria.
“Reward system” refers to a group of neurobiological mechanisms that are induced by rewarding stimuli such as, e.g., food, drug or alcohol. The reward system involves several structures in the brain, including the limbic system, the basal ganglia, the prefrontal cortex, the ventral tegmental area, the striatum, the nucleus accumbens, and the substantia nigra. The activation of the reward system by the anticipation or acquisition of a reward induces a positive or pleasurable sensation in the individual, which stems from the release of the neurotransmitter dopamine, or other neurotransmitters such as GABA, glutamate, serotonin, and norepinephrine, but also from the release of opioids and/or endocannabinoids. Certain drugs are capable of activating directly the reward system without a rewarding stimulus. Importantly, the reward system comprises 3 components: the “liking” component, the “wanting” component, and the “learning” component. The liking component corresponds to the hedonic impact, and is related to the pleasurable sensation provided by the rewarding stimulus. The wanting component corresponds to incentive salience, and is related to the motivation or incentive that an individual gets in order to obtain the reward. The learning component corresponds to the ability of the individual to perform predictive associations between the reward and a context (e.g., a place, a time of day, an action or sequence of actions, and the like), and to durably memorize this association for future acquisitions of rewards.
“Reward dysregulation disorders” refers to disorders wherein the individual is striving to pursue or attain pleasurable stimuli, and anticipatory pleasure; and/or experiences heightened response to positive or reward-laden cues, or positive emotion reactivity (see Gruber et al., J Abnorm Child Psychol. 2013; 41 (7): 1053-1065), or wherein the individual necessitates higher levels of exposure to the reward in order to attain the same pleasure. In some embodiments, any one of the 3 components of the reward system (i.e., the liking, wanting and learning components) may be dysregulated. In some embodiments, 1, 2 or all of the 3 components are dysregulated. By “dysregulated”, it is meant that a component may be either abnormally “over-stimulated” (i.e., activated, overactivated, increased, upregulated) or “under-stimulated” (i.e., inhibited, decreased, downregulated). In one embodiment, one or more components are over-stimulated or under-stimulated. In another embodiment, one or more components are over-stimulated and one or more distinct components are under-stimulated. In practice, reward dysregulation disorders encompass mental disorders and neurological disorders, which are defined below. Diagnosis of individuals with reward dysregulation disorders may be performed by authorized personnel, such as a physician, accordingly to the standards protocols in the field, in particular by monitoring clinical signs, and often with the assistance of a questionnaire.
“Mental disorders”, as used herein, represents a particular subset of reward dysregulation disorders, and refers to disorders that are characterized by a combination of abnormal thoughts, perceptions, emotions, behavior and relationships with others, as defined by the World Health Organization (WHO). In practice, mental disorders include addiction-related disorders, eating-related disorders, affective disorders, obsessive compulsive disorders, schizophrenia, attention deficit hyperactivity disorders (ADHD), autism spectrum disorder, major depressive disorder (MDD), anxiety disorder, and the like. In some embodiments, mental disorders comprise eating-related disorder and addiction-related disorder.
“Eating-related disorders” refers to a particular subset of mental disorders, and comprises anorexia, bulimia, overweight-related disorders, obesity-related disorders, and the like. In some embodiments, “overweight-related disorders” and “obesity-related disorders” are used interchangeably, and refer to disorders related to a body mass index (BMI) greater than or equal to 25 (for overweight) or to a BMI greater than or equal to 30 (for obesity), as defined by the WHO. Within the scope of the present invention, “overweight-related disorders” and “obesity-related disorders” are associated with abnormal food intake, inducing and/or maintaining a BMI greater than or equal to 25 or 30.
“Addiction-related disorders” refers to a particular subset of mental disorders, and comprises alcohol-related addiction, drug-related addiction, tobacco or nicotine addiction, game-related addiction, and the like.
“Neurological disorders”, as used herein, represents a particular subset of reward dysregulation disorders, and refers to disorders that affect the brain, the nerves and the spinal cord. In practice, individuals with neurological disorders may experience symptoms such as, e.g., paralysis, muscle weakness, poor coordination, loss of sensation, seizures, confusion, pain and altered levels of consciousness. Non-limitative examples of neurological disorders include neuromuscular disorders, autism spectrum disorders, neurodegenerative disorders (e.g., Alzheimer's disease, Parkinson's disease), Tourette Syndrome, epilepsy, amyotrophic lateral sclerosis.
“Beneficial microbes” refers to microorganisms that may provide health benefits to the hosts, including improvement of the host intestinal microbial balance, maintaining the intestinal gut barrier homeostasis, preventing pathogen colonization, preventing bacterial and viral infections.
“Prevention” refers to preventing or avoiding the occurrence of symptom of a reward dysregulation disorder. In the present invention, the term “prevention” may refer to a secondary prevention, i.e., to the prevention of the re-occurrence of a symptom or a relapse of a reward dysregulation disorder.
“Treating” or “treatment” or “alleviation” refers to both therapeutic treatment and prophylactic or preventative measures, wherein the object is to prevent or slow down (lessen) the targeted reward dysregulation disorder. Those in need of treatment include those already with the reward dysregulation disorder as well as those prone to have the reward dysregulation disorder or those in whom the reward dysregulation disorder is to be prevented. An individual or mammal is successfully “treated” for a reward dysregulation disorder or condition, if, after receiving a therapeutic amount of a composition, pharmaceutical composition, according to the present invention, alone or in combination with another treatment, the patient shows observable and/or measurable reduction in, or absence of, one or more of the symptoms associated with the reward dysregulation disorder; and/or relief to some extent, one or more of the symptoms associated with the reward dysregulation disorder or condition; reduced morbidity and mortality, and improvement in quality of life issues. The above parameters for assessing successful treatment and improvement in the disease are readily measurable by routine procedures familiar to a physician.
“Therapeutically effective amount” refers to an amount sufficient to effect beneficial or desired results including clinical results. A therapeutically effective amount can be administered in one or more administrations. In one embodiment, the therapeutically effective amount may depend on the individual to be treated.
“Pharmaceutically acceptable carrier” refers to a carrier that does not produce any adverse, allergic or other unwanted reactions when administered to an animal individual, preferably a human individual. It includes any and all solvents, dispersion media, coatings, antibacterial and antifungal agents, isotonic and absorption delaying agents and the like. For human administration, preparations should meet sterility, pyrogenicity, general safety, quality and purity standards as required by regulatory Offices, such as, e.g., the Food and Drug Administration (FDA) in the United States or the European Medicines Agency (EMA) in the European Union.
“Individual” refers to an animal individual, preferably a mammalian individual, more preferably a human individual. In some embodiments, an individual may be a mammalian individual. Mammalians include, but are not limited to, all primates (human and non-human), cattle (including cows), horses, pigs, sheep, goats, dogs, cats, and any other mammal which is awaiting the receipt of, or is receiving medical care or was/is/will be the object of a medical procedure, or is monitored for the development of a reward dysregulation disorder. In some embodiments, an individual may be a “patient”, i.e., a warm-blooded animal, more preferably a human, who/which is awaiting the receipt of, or is receiving medical care or was/is/will be the object of a medical procedure, or is monitored for the development of a reward dysregulation disorder. In some embodiments, the individual is an adult (e.g., an individual above the age of 18). In some embodiments, the individual is a child (e.g., an individual below the age of 18). In some embodiments, the individual is a male. In some embodiments, the individual is a female.

Other definitions may appear in context throughout this disclosure.

### DETAILED DESCRIPTION

This invention relates to a composition comprising one or more bacteria from the genus Parabacteroides and/or extracts thereof, for use in preventing and/or treating reward dysregulation disorders.

In some aspects, the invention also relates to the use of a composition comprising one or more bacteria from the genus Parabacteroides and/or extracts thereof, for preventing and/or treating reward dysregulation disorders.

The invention further pertains to the use of a composition comprising one or more bacteria from the genus Parabacteroides and/or extracts thereof, for the preparation or the manufacture of a medicament for preventing and/or treating reward dysregulation disorders.

In another aspect, the invention relates to a method for preventing and/or treating reward dysregulation disorders in an individual in need thereof, comprising the administration of a therapeutically effective amount of a composition comprising one or more bacteria from the genus Parabacteroides and/or extracts thereof.

According to some embodiments, the bacteria from the genus Parabacteroides are selected in the group comprising or consisting of P. distasonis, P. goldsteinii, P. merdae, P. acidifaciens, P. bouchesdurhonensis, P. chartae, P. chinchilla, P. chongii, P. faecis, P. gordonii, P. johnsonii, P. massiliensis, P. pacaensis, P. provencensis, P. timonensis, Parabacteroides spp. and combinations thereof.

In some embodiments, the bacteria from the genus Parabacteroides are selected in the group comprising or consisting of P. distasonis, P. goldsteinii and P. merdae. In some embodiments, the bacteria from the genus Parabacteroides are P. distasonis or P. goldsteinii. In some embodiments, the bacteria from the genus Parabacteroides are P. goldsteinii. In some embodiments, the bacteria from the genus Parabacteroides are P. distasonis. In some embodiments, the bacteria from the genus Parabacteroides are P. merdae.

In practice, bacteria belonging to the genus Parabacteroides may be identified by any suitable procedures, or a procedure adapted therefrom. In particular, suitable procedures may include physiological and biochemical methods, such as the assessment of the capacity to ferment on selected nutrients, e.g., mannose, raffinose; the assessment of the resistance to some antibiotics; the assessment of specific enzymatic activities, such as, e.g., alpha-galactosidase, beta-galactosidase, alpha-glucuronidase, alkaline phosphatase, L-arginine arylamidase, Leucine glycine arylamidase, Phenylalanine arylamidase; the assessment of their cellular fatty acid profiles, menaquinone profiles; the assessment of their profile by matrix-assisted laser-desorption/ionization time-of-flight mass spectrometry (MALDI-TOF MS); the assessment of their phylogenetic position, based on 16S rRNA gene sequence analysis.

In some embodiments, the bacteria from the genus Parabacteroides are isolated. In some embodiments, the bacteria from the genus Parabacteroides are isolated from a natural habitat, such as, e.g., the gut microbiota. In practice, the bacteria from the genus Parabacteroides may be isolated from feces or ceacal content, fresh or frozen, diluted or not in a specific medium (including cryoprotectants and/or antioxidants), accordingly to the standard and ethical procedures in the field.

In practice, bacteria from the genus Parabacteroides may be cultured in any suitable culture medium, such as, e.g., the Yeast Casitone Fatty Acids (YCFA) (commercially available from Fisher Scientific®), the Columbia blood medium (commercially available from Sigma Aldrich®, DSMZ®), the fastidious anaerobe broth (commercially available from DSMZ®, Neogen®), the chopped meat medium with carbohydrates (commercially available from DSMZ®), or a modified YCFA medium wherein myo-inositol is replaced by glucose.

In practice, cultures of bacteria from the genus Parabacteroides may be performed at a temperature ranging from about 30° C. to about 42° C., preferably from about 35° C. to about 40° C., more preferably at about 37° C. As used herein, the term “about 30° C. to about 42° C.” includes about 30° C., 31° C., 32° C., 33° C., 34° C., 35° C., 36° C., 37° C., 38° C., 39° C., 40° C., 41° C. and 42° C.

In practice, cultures of bacteria from the genus Parabacteroides may be performed in anaerobic conditions, i.e., in the absence of O₂.

In some embodiments, the composition of the invention comprises or substantially consists of a microbiota with bacteria from the genus Parabacteroides obtained from an individual. In one embodiment, the microbiota is a gut microbiota obtained from the feces of an individual. In one embodiment, the microbiota is enriched with bacteria from the genus Parabacteroides compared to the microbiota of the individual to be treated.

In some embodiments, the composition of the invention is enriched with bacteria from the genus Parabacteroides. In one embodiment, the composition of the invention comprises or substantially consists of a microbiota enriched with bacteria from the genus Parabacteroides.

In practice, bacteria from the genus Parabacteroides may be enriched by preferentially stimulating the growth of the bacteria from the genus Parabacteroides. For example, enrichment may be performed by modifying physiological conditions of the culture. Examples include, but are not limited to, modification of the composition of the culture media, such as the nutrient composition; and modification of the culture conditions, such as environmental pH value, temperature and oxygen conditions, and the like.

In some embodiments, the bacteria from the genus Parabacteroides are isolated and enriched. In some embodiments, the composition of the invention comprises isolated, enriched bacteria from the genus Parabacteroides.

In one embodiment, the bacteria from the genus Parabacteroides are viable. As used herein, the term “viable” refers to bacteria that are able to maintain an active metabolism and/or proliferate in a suitable culture medium, under suitable culture conditions, including suitable pH, temperature, salinity, nutrients content, O₂ content. In some embodiments, the bacteria from the genus Parabacteroides are in long-lasting exponential growth phases and/or stationary growth phase.

In one embodiment, the bacteria from the genus Parabacteroides are non-viable. As used herein, the term “non-viable” refers to bacteria that are not able to maintain an active metabolism and/or proliferate in a suitable culture medium, under suitable culture conditions, including suitable pH, temperature, salinity, nutrients content, O₂ content. Example of non-viable bacteria are dormant bacteria, dead bacteria and inactive bacteria.

In practice, cell viability (active metabolism) may be assessed by measuring the consumption of one nutrient in the culture medium over time. Cell viability (proliferation) may be assessed by spreading a solution containing at least one bacterium of the invention across a petri dish and counting the number of colonies after a determined time of incubation in suitable culture conditions; alternatively, bacteria may be grown in liquid medium, and proliferation may be measured by measuring optical density of the bacterial culture after a determined time of incubation in suitable culture conditions.

In one embodiment, the bacteria from the genus Parabacteroides are pasteurized. In one embodiment, the pasteurized Parabacteroides and/or extracts thereof were heated at a temperature ranging from about 50° C. to about 100° C., preferably from about 60° C. to about 95° C., more preferably from about 70° C. to about 90° C.

In some embodiments, the bacteria from the genus Parabacteroides are pasteurized Parabacteroides distasonis, pasteurized Parabacteroides goldsteinii or pasteurized Parabacteroides merdae. In some embodiments, the bacteria from the genus Parabacteroides are pasteurized Parabacteroides goldsteinii. In some embodiments, the bacteria from the genus Parabacteroides are pasteurized Parabacteroides distasonis. In some embodiments, the bacteria from the genus Parabacteroides are pasteurized Parabacteroides merdae.

As used herein, the term “extracts” encompasses both cellular and extracellular extracts.

In practice, cellular extracts include cytoplasmic extracts, membrane extracts, and combination thereof, in particular, extracts obtained from fractionation methods. Cellular extracts may be obtained by any standard chemical (implementing SDS, proteinase K, lysozyme, combinations thereof, and the like) and/or mechanical (sonication, pressure) fractionation approaches, or approaches adapted therefrom.

In practice, extracellular extracts may include the secreted fraction, in particular soluble compounds or exosomes. As used herein, the term “exosomes” is intended to refer to endocytic-derived nanovesicles that comprise proteins, nucleic acids, and lipids. In practice, the secreted fraction may be isolated and/or purified from the culture medium, according to any suitable method known in the state of the art, or a method adapted therefrom. Illustratively, the extracellular extracts may be isolated by differential centrifugation from culture medium; by polymer precipitation; by high-performance liquid chromatography (HPLC), combination thereof, and the like.

Non-limitative example of differential centrifugation method from culture medium may include the following steps:
centrifugation for 10-20 min at a speed of about 300×g to about 500×g, so as to remove cells;
centrifugation for 10-20 min at a speed of about 1,500×g to about 3,000×g, so as to remove dead cells;
centrifugation for 20-45 min at a speed of about 7,500×g to about 15,000×g, so as to remove cell debris;
one or more ultracentrifugation for 30-120 min at a speed of about 100,000×g to about 200,000×g, so as to pellet the exosomes.

Alternative methods to isolate exosomes may take advantage of commercial kits, such as, e.g., the exoEasy Maxi Kit (Qiagen®) or the Total Exosome Isolation Kit (Thermo Fisher Scientific®).

In practice, cellular and/or extracellular extracts may comprise nucleic acids, proteins, carbohydrates, lipids and combinations of these such as lipoproteins, glycolipids and glycoproteins, bacterial metabolites, organic acids, inorganic acids, bases, peptides, enzymes and co-enzymes, amino acids, carbohydrates, lipids, glycoproteins, lipoproteins, glycolipids, vitamins, bioactive compounds, metabolites such as metabolites containing an inorganic component, and the like.

In some embodiments, the cellular and/or extracellular extracts are produced during the long-lasting exponential growth phases and/or the stationary growth phase.

In some embodiments, the cellular extract comprises succinate. In some embodiments, the metabolite is succinate. Accordingly, an object of the present invention is a composition comprising succinate for use in preventing and/or treating reward dysregulation disorders.

It is to be understood that the reward dysregulation disorders according to the invention may be diagnosed and/or monitored through the evaluation of clinical signs, with or without the assistance of a dedicated questionnaire. In practice, the diagnosis and/or monitoring of reward dysregulation disorders may be performed by authorized personnel.

The reward system comprises at least 3 components: the “liking” component, the “wanting” component, and the “learning” component. It is to be understood that any one of the 3 components of the reward system may be dysregulated. In some embodiments, 1, 2 or all of the 3 components are dysregulated.

As used herein, “dysregulated” means that a component is abnormally “over-stimulated” (i.e., activated, overactivated, increased, upregulated) or abnormally “under-stimulated” (i.e., inhibited, less activated, decreased, downregulated).

In one embodiment, one or more components are over-stimulated or under-stimulated. In certain embodiments, one component is over-stimulated or under-stimulated. In certain embodiments, two components are over-stimulated or under-stimulated. In certain embodiments, three components are over-stimulated or under-stimulated.

In certain embodiments, the wanting component is over-stimulated. In certain embodiments, the liking component is over-stimulated. In certain embodiments, the liking component is under-stimulated. In certain embodiments, the wanting component is under-stimulated. In certain embodiments, the learning component is over-stimulated. In certain embodiments, the learning component is under-stimulated. In certain embodiments, the liking and wanting components are over-stimulated. In certain embodiments, the liking and wanting components are under-stimulated. In certain embodiments, the liking and learning components are over-stimulated. In certain embodiments, the liking and learning components are under-stimulated. In certain embodiments, the wanting and learning components are over-stimulated. In certain embodiments, the wanting and learning components are under-stimulated. In certain embodiments, all three components are over-stimulated. In certain embodiments, all three components are under-stimulated.

In another embodiment, one or more components are over-stimulated and one or more distinct components are under-stimulated. In certain embodiments, one component is over-stimulated and two components are under-stimulated. In certain embodiments, one component is under-stimulated and two components are over-stimulated. In certain embodiments, one component is over-stimulated and one component is under-stimulated.

In certain embodiments, the liking component is under-stimulated and the wanting component is over-stimulated. In certain embodiments, the liking component is over-stimulated and the wanting component is under-stimulated. In certain embodiments, the liking component is under-stimulated and the learning component is over-stimulated. In certain embodiments, the liking component is over-stimulated and the learning component is under-stimulated. In certain embodiments, the wanting component is under-stimulated and the learning component is over-stimulated. In certain embodiments, the wanting component is over-stimulated and the learning component is under-stimulated.

In certain embodiments, the liking and learning components are under-stimulated and the wanting component is over-stimulated. In certain embodiments, the liking and learning components are over-stimulated and the wanting component is under-stimulated. In certain embodiments, the liking and wanting components are under-stimulated and the learning component is over-stimulated. In certain embodiments, the liking and wanting components are over-stimulated and the learning component is under-stimulated. In certain embodiments, the wanting and learning components are under-stimulated and the liking component is over-stimulated. In certain embodiments, the wanting and learning components are over-stimulated and the liking component is under-stimulated.

In some embodiments, the composition for use according to the invention restores an over-stimulated or under-stimulated component to a normal level. In some embodiments, the composition for use according to the invention decreases at least one over-stimulated component. In some embodiments, the composition for use according to the invention increases at least one under-stimulated component.

According to certain embodiments, the reward dysregulation disorder is selected in a group comprising or consisting of mental disorders, neurological disorders, and combinations thereof.

In some embodiments, the reward dysregulation disorder is a mental disorder. Mental disorders or mental illness, also called mental health disorders, refers to a wide range of mental health conditions, i.e., disorders that affect mood, thinking and behavior. Examples of mental illness include depression, anxiety disorders, schizophrenia, eating-related disorders, obsessive compulsive behaviors and addictive behaviors.

According to some embodiments, the mental disorder is selected in a group comprising or consisting of addiction-related disorder, eating-related disorder, affective disorders, obsessive compulsive disorders, schizophrenia, attention deficit hyperactivity disorders (ADHD), autism spectrum disorder, major depressive disorder (MDD), anxiety disorder, and the like. According to some embodiments, the mental disorder is selected in a group comprising or consisting of addiction-related disorders, eating-related disorders and obsessive compulsive disorders. According to one embodiment, the mental disorder is selected in a group comprising or consisting of addiction-related disorders and eating-related disorders.

In some embodiments, the mental disorder is an eating-related disorder.

According to certain embodiments, the eating-related disorder is selected in a group comprising or consisting of anorexia, bulimia, binge eating, overweight-related disorders, obesity-related disorders, and the like.

As used herein, an individual with overweight-related disorder has a body mass index (BMI) comprised from about 25.0 to about 29.9. As used herein, an individual with obesity-related disorder has a body mass index (BMI) above about 30.0.

In one embodiment, the eating-related disorder is anorexia. In one embodiment, the eating-related disorder is bulimia. In one embodiment, the eating-related disorder is binge eating. As used herein, “binge eating”, also referred to as “binge eating disorder” refers to an abnormal behavior comprising compulsive food intake, overeating and/or food addiction; binge eating may be associated with bulimia. In one embodiment, the eating-related disorder is overweight-related disorder or obesity-related disorder. In one embodiment, the eating-related disorder is overweight-related disorder. In one embodiment, the eating-related disorder is obesity-related disorder.

It is to be understood that a subject suffering from an eating-related disorder may have reduced pleasure in eating food due to an under-stimulation of the liking component of the reward system, and a dysregulation of the wanting component of the reward system. The dysregulation of the wanting component may be an over-stimulation or an under-stimulation, which may lead to excessive or insufficient food intake. The dysregulation of the wanting component may partially be involved in diseases such as binge eating and anorexia.

In some embodiments, the eating-related disorder is associated with a dysregulation of the wanting component of the reward system. In some embodiments, the eating-related disorder is associated with an over-stimulation of the wanting component of the reward system, preferably the eating-related disorder is associated with an over-stimulation of the wanting component and an under-stimulation of the liking component of the reward system. In some embodiments, the eating-related disorder is induced by an over-stimulation of the wanting component of the reward system, preferably the eating-related disorder is induced by an over-stimulation of the wanting component and an under-stimulation of the liking component of the reward system.

In one embodiment, binge eating is associated with an over-stimulation of the wanting component of the reward system.

In some embodiments, the mental disorder is an addiction-related disorder.

According to some embodiments, the addiction-related disorder is selected in a group comprising or consisting of alcohol-related addiction, drug-related addiction, tobacco or nicotine addiction, game-related addiction, and the like.

According to some embodiments, the obsessive compulsive disorders (OCD) is selected in a group comprising or consisting of checking OCD, contamination OCD, counting OCD, harm OCD, hoarding OCD, perinatal OCD, postpartum OCD, and the like.

In some embodiments, OCD and eating-related disorders occur concomitantly.

In some embodiments, OCD abnormally increases or decreases the appetence of an individual for certain types of food or aliments, wherein “appetence” reflects the wanting and/or liking components of the reward system of the individual.

In some embodiments, the reward dysregulation disorder is a neurological disorder.

According to certain embodiments, the neurological disorder is selected in a group comprising or consisting of Parkinson's disease, Tourette Syndrome, and the like.

In some embodiments, the neurological disorder comprises a dysregulation of the neurotransmitter dopamine, wherein “dysregulation” means altered signaling, altered expression of dopaminergic markers, altered levels, altered recycling or combinations thereof.

According to some embodiments, the composition is to be administered to an animal individual, preferably a mammalian individual, more preferably a human individual.

In one embodiment, the individual is a mammalian individual. In one embodiment, the individual is a human individual. In one embodiment the individual is a male. In one embodiment, the individual is a female.

According to certain embodiments, the composition is to be administered orally or rectally.

In one embodiment, the composition is administered into the digestive tract. It is to be understood that the digestive tract is the final location of the bacteria according to the invention. In other words, the bacteria according to the invention are intended to be incorporated into the microbiota of the individual.

In one embodiment, the composition is a solid composition. In practice, solid forms adapted to oral administration include, but are not limited to, pill, tablet, capsule, soft gelatin capsule, hard gelatin capsule, dragees, granules, gums, chewing gums, caplet, compressed tablet, cachet, wafer, sugar-coated pill, sugar coated tablet, or dispersing/or disintegrating tablet, powder, solid forms suitable for solution in, or suspension in, liquid prior to oral administration and effervescent tablet.

In one embodiment, the composition is a liquid composition. In practice, liquid form adapted to oral administration include, but are not limited to, solutions, suspensions, drinkable solutions, elixirs, sealed phial, potion, drench, syrup, liquor and sprays.

According to some embodiments, the bacteria are to be administered at a dose comprised from about 1×10² CFU/g to about 1×10¹² CFU/g of the composition, preferably from about 1×10³ CFU/g to about 1×10¹¹ CFU/g of the composition, more preferably from about 1×10⁴ CFU/g to about 1×10¹⁰ CFU/g of the composition. In one embodiment, the bacteria are to be administered at a dose comprised from about 1×10⁴ CFU/g to about 1×10¹¹ CFU/g of the composition, from about 1×10⁵ CFU/g to about 1×10¹¹ CFU/g of the composition, from about 1×10⁶ CFU/g to about 1×10¹¹ CFU/g of the composition, from about 1×10⁷ CFU/g to about 1×10¹¹ CFU/g of the composition or from about 1×10⁸ CFU/g to about 1×10¹¹ CFU/g of the composition.

As used herein, “CFU” stands for “Colony Forming Unit”. As used herein the term “about 1×10² CFU/g to about 1×10¹² CFU/g” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² CFU/g.

According to some embodiments, the bacteria are to be administered at a dose comprised from about 1×10² cells/g to about 1×10¹² cells/g of the composition. As used herein the term “about 1×10² cells/g to about 1×10¹² cells/g” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² cells/g.

According to some embodiments, when the composition is a solid composition, the bacteria are to be administered at a dose comprised from about 1×10² CFU/g to about 1×10¹² CFU/g of the composition. As used herein the term “about 1×10² CFU/g to about 1×10¹² CFU/g” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² CFU/g.

According to some embodiments, when the composition is a solid composition, the bacteria are to be administered at a dose comprised from about 1×10² cells/g to about 1×10¹² cells/g of the composition. As used herein the term “about 1×10² cells/g to about 1×10¹² cells/g” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² cells/g.

According to some embodiments, when the composition is a liquid composition, the bacteria are to be administered at a dose comprised from about 1×10² CFU/ml to about 1×10¹² CFU/ml of the composition. As used herein the term “about 1×10² CFU/ml to about 1×10¹² CFU/ml” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² CFU/ml.

According to some embodiments, when the composition is a liquid composition, the bacteria are to be administered at a dose comprised from about 1×10² cells/ml to about 1×10¹² cells/ml of the composition. As used herein the term “about 1×10² cells/ml to about 1×10¹² cells/ml” includes 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹ and 1×10¹² cells/ml.

The present invention further relates to a composition comprising succinate, for use in preventing and/or treating reward dysregulation disorders. Reward dysregulation disorders have been described hereinabove.

In some embodiments, the succinate is produced by bacteria from the genus Parabacteroides. In some embodiments, the succinate is produced by Parabacteroides distasonis, Parabacteroides goldsteinii or Parabacteroides merdae.

In some embodiments, the succinate is administered to the subject in a therapeutically effective amount.

By “therapeutically effective amount”, it is meant a level or amount that is necessary and sufficient for preventing, slowing down or stopping the progression, aggravation, or deterioration of one or more symptoms of at least one reward dysregulation disorder as defined herein; or alleviating the symptoms of at least one reward dysregulation disorder; or curing at least one reward dysregulation disorder, without causing significant negative or adverse side effects to the individual. In certain embodiments, an effective amount of succinate may range from about 0.001 mg to about 3,000 mg, per dosage unit.

Within the scope of the instant invention, from about 0.001 mg to about 3,000 mg includes, from about 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg and 3,000 mg per dosage unit.

In certain embodiments, the succinate is to be administered at dosage levels sufficient to deliver from about 0.001 mg/kg to about 100 mg/kg of subject body weight per day.

The present invention further relates to a method for preventing and/or treating reward regulation disorders, comprising administering to a subject in need thereof a therapeutically effective amount of a composition comprising succinate. In certain embodiments, an effective amount of succinate may range from about 0.001 mg to about 3,000 mg, per dosage unit.

The present invention further relates to a composition comprising succinate for use in the manufacture of a medicament for the treatment and/or the prevention of reward regulation disorders.

According to certain embodiments, the composition of the invention further comprises one or more additional active agent(s).

According to certain embodiments, the one or more additional active agent(s) are one or more beneficial microbe(s). In other words, in one embodiment, the composition further comprises one or more beneficial microbe(s).

According to some embodiments, the one or more beneficial microbe(s) is/are selected in a group comprising or consisting of bacteria from the family Clostridiaceae, from the family Peptostreptococcaceae, from the family Prevotellaceae, from the family Methylobacteriaceae, from the genus Turicibacter, from the genus Coprococcus, from the genus Knoellia, from the genus Prevotella, from the genus Staphylococcus, from the genus Akkermansiaceae, and the like.

According to some embodiments, the one or more beneficial microbe(s) is/are selected in a group comprising or consisting of bacteria from the family Clostridiaceae, from the family Peptostreptococcaceae, from the family Prevotellaceae, from the family Methylobacteriaceae, from the genus Turicibacter, from the genus Coprococcus, from the genus Knoellia, from the genus Prevotella, from the genus Staphylococcus, and the like.

According to certain embodiments, the composition is in the form of a pharmaceutical composition further comprising a pharmaceutically acceptable carrier.

In certain embodiments, pharmaceutically acceptable carriers that may be used in the pharmaceutical compositions according to the invention include, but are not limited to, ion exchangers; alumina; aluminum stearate; lecithin; serum proteins, such as human serum albumin; buffer substances such as phosphates; glycine; sorbic acid; potassium sorbate; partial glyceride mixtures of vegetable oil saturated fatty acids; water; salts or electrolytes, such as protamine sulfate, disodium hydrogen phosphate, potassium hydrogen phosphate, sodium chloride, zinc salts; colloidal silica; magnesium trisilicate, polyvinyl pyrrolidone; cellulose-based substances (e.g., sodium carboxymethyl cellulose), polyethylene glycol; polyacrylates; waxes; polyethylene-polyoxypropylene-block polymers; polyethylene glycol; wool fat; the like; and any combination thereof.

According to certain embodiments, the composition is in the form of a nutritional composition further comprising a nutritionally acceptable carrier.

As used herein, the term “nutritional composition” is intended to refer to any food product, additive food, supplement food, fortified food, including liquid food products and solid food products. In practice, liquid food products include, but are not limited to, soups, soft drinks, sports drinks, energy drinks, fruit juices, lemonades, teas, milk-based drinks, and the like. In practice, solid food products include, but are not limited to candy bars, cereal bars, energy bars, and the like.

In some embodiments, the nutritional composition of the invention is for non-therapeutic use, or for use in a non-therapeutic method.

In some aspects, the invention relates to a medicament comprising a therapeutically effective amount of one or more isolated bacteria from the genus Parabacteroides and/or extracts thereof, for use in preventing and/or treating reward dysregulation disorders.

In some embodiments, the composition, the pharmaceutical composition, the nutritional composition, the medical device or the medicament according to the invention is sterile. In practice, methods for obtaining a sterile pharmaceutical composition include, but are not limited to, GMP synthesis (GMP stands for “Good manufacturing practice”).

The present invention also relates to a medical device comprising, consisting of, or consisting essentially of one or more isolated bacteria from the genus Parabacteroides and/or extracts thereof, for use in preventing and/or treating reward dysregulation disorders. In one embodiment, the medical device according to the invention comprises a therapeutically effective amount of one or more isolated bacteria from the genus Parabacteroides and/or extracts thereof.

According to certain embodiments, the composition is comprised in a kit, which further comprises means to administer said composition.

The present invention also relates to a composition comprising one or more active ingredients or substances that increase the level of bacteria from the genus Parabacteroides in the microbiota of an individual in need thereof. As used herein, “increasing the level of bacteria from the genus Parabacteroides in the microbiota” means increasing the relative abundance of bacteria from the genus Parabacteroides in the microbiota of the individual after administration of the composition of the invention, compared to the relative abundance of bacteria from the genus Parabacteroides in the microbiota of the individual before administration of the composition of the invention.

The present invention further relates to a method for restoring the reward function in an individual in need thereof. In one embodiment, the method comprises the administration of a composition comprising one or more active ingredients or substances that increase the level of bacteria from the genus Parabacteroides in the microbiota. In a particular embodiment, the method comprises the administration of a composition comprising one or more bacteria from the genus Parabacteroides and/or extracts thereof. In another embodiment, the method comprises the administration of a composition comprising succinate. In one embodiment, this method is non-therapeutic.

The present invention further relates to a method for restoring the microbiota of an individual in need thereof. In one embodiment, the method comprises the administration of a composition comprising one or more active ingredients or substances that increase the level of bacteria from the genus Parabacteroides in the microbiota. In a particular embodiment, the method comprises the administration of a composition comprising one or more bacteria from the genus Parabacteroides and/or extracts thereof. In one embodiment, this method is non-therapeutic.

The present invention further relates to a method for increasing the level of Parabacteroides in the microbiota of an individual in need thereof. In one embodiment, the method comprises the administration of a composition comprising one or more active ingredients or substances that increase the level of bacteria from the genus Parabacteroides in the microbiota. In a particular embodiment, the method comprises the administration of a composition comprising one or more bacteria from the genus Parabacteroides and/or extracts thereof. In one embodiment, this method is non-therapeutic.

The present invention also relates to a method for reducing the reward eating in an individual in need thereof. In one embodiment, the method comprises the administration of a composition comprising one or more active ingredients or substances that increase the level of bacteria from the genus Parabacteroides in the microbiota. In a particular embodiment, the method comprises the administration of a composition comprising one or more bacteria from the genus Parabacteroides and/or extracts thereof. In another embodiment, the method comprises the administration of a composition comprising succinate. In one embodiment, this method is non-therapeutic. In some embodiments, the method reduces the intake of palatable food. In some embodiments, the method does not reduce the intake of palatable food.

The present invention further relates to a method for reducing the intake of palatable diet in an individual in need. In one embodiment, the method comprises the administration of a composition comprising one or more active ingredients or substances that increase the level of Parabacteroides in the microbiota. In a particular embodiment, the method comprises the administration of a composition comprising one or more bacteria from the genus Parabacteroides and/or extracts thereof. In another embodiment, the method comprises the administration of a composition comprising succinate. In one embodiment, this method is non-therapeutic.

The present invention further relates to a method for modulating the reward function in an individual in need thereof, comprising administering to the individual a composition comprising one or more bacteria from the genus Parabacteroides and/or extracts thereof.

As used herein, “modulating the reward function” means increasing or decreasing the activity of at least one of the three components of the reward system (i.e., liking, wanting and learning), so that the at least one component is restored to normal levels. In some embodiments, one component is modulated. In some embodiments, two components are modulated. In some embodiments, three components are modulated.

In some embodiments, the method is for modulating the wanting component. In some embodiments, the method is for increasing or decreasing the wanting component. In a preferred embodiment, the method is for decreasing the wanting component. In another embodiment, the method is for increasing the wanting component.

In some embodiments, the method is for modulating the liking component. In some embodiments, the method is for increasing or decreasing the liking component. In a preferred embodiment, the method is for increasing the liking component. In another embodiment, the method is for decreasing the liking component.

In some embodiments, the method is for modulating the learning component. In some embodiments, the method is for increasing or decreasing the learning component.

In one embodiment, the method is for decreasing the learning component. In another embodiment, the method is for increasing the learning component.

Other objects of the present invention are the methods as described hereinabove comprising administering to the individual a composition comprising succinate.

### BRIEF DESCRIPTION OF THE DRAWINGS

FIGS. 1A-C are a set of graphs showing that obese mice present a reduced food preference for high fat, high sucrose (HFHS) compared to lean mice. (FIG. 1A) Body weight evolution (in grams) of lean (Lean_do; squares) and DIO donor mice (DIO_do; triangles) and (FIG. 1B) final body weight (in grams), after a 5 weeks period. (FIG. 1C) Fat mass gain evolution (in grams) of lean (Lean_do; squares) and DIO donor mice (DIO_do; triangles) and (FIG. 1D) final fat mass gain (in grams). (FIG. 1E) Food preference test showing HFHS and CT intake during 180 minutes of test by lean (Lean_do) and DIO donor mice (DIO_do). (FIG. 1F) Food preference test showing total HFHS and CT intake, from FIG. 1E. Data are shown as mean±SEM (n=5/group). P-values were obtained after Two-way ANOVA, followed by Bonferroni post-hoc test (FIG. 1A, C, E, F), unpaired Student's t-test (FIG. 1B, D). *: p-value≤0.05; **: p-value≤0.01; ***: p-value≤0.001; ****: p-value≤0.0001. $$$$: p-value≤0.0001 between CT vs HFHS intake. Different superscript letters represent significant p-values between groups and type of diet (CT or HFHS) at each time-point (FIG. 1F).

FIGS. 2A-G are a set of graphs showing that recipient mice show hedonic food behaviour similar to donor mice after fecal transplantation. (FIG. 2A) Experimental plan of the FMT protocol. (FIG. 2B) Body weight evolution (in grams) and (FIG. 2C) final body weight (in grams), of lean (Lean_rec; squares) and DIO recipient mice (DIO_rec; triangles). (FIG. 2D) Fat mass gain evolution (in grams) and (FIG. 2E) final fat mass gain (in grams), of lean (Lean_rec; squares) and DIO recipient mice (DIO_rec; triangles). (FIG. 2F) Food preference test showing total HFHS and CT intake after 180 minutes of test by lean (Lean_rec) and DIO recipient mice (DIO_rec). Data are shown as mean±SEM (n=7-8/group). (FIG. 2G) Food preference test showing HFHS (curves 3 and 4) and CT (curves 1 and 2) intake during 180 minutes by lean (Lean_rec; curves 1 and 3) and DIO recipient mice (DIO_rec; curves 2 and 4). P-values were obtained after Two-way ANOVA, followed by Bonferroni post-hoc test (FIG. 2B, D, F, G) or unpaired Student t-test (FIG. 2C, E). *: p-value≤0.05; **: p-value≤0.01. $$: p-value<0.01; $$$$: p-value≤0.0001 between CT vs HFHS intake (FIG. 2F).

FIGS. 3A-D are a set of graphs showing alterations in dopaminergic signaling in recipient mice with obese gut microbiota. Striatal mRNA expression of dopamine receptor 1 (DIR) (FIG. 3A), dopamine receptor 2 (D2R) (FIG. 3B), tyrosine hydroxylase (TH) (FIG. 3C) and dopamine transporter (DAT) (FIG. 3D) measured by real-time qPCR in lean (Lean_rec) and DIO recipient mice (DIO_rec). Data are shown as mean±SEM (n=7-8/group). P-values were obtained after unpaired Student's t-test (FIG. 3C) or non-parametric Mann-Whitney test (FIG. 3A, B, D).

FIGS. 4A-F are a set of graphs showing that the gut microbiota of recipient mice is similar to the gut microbiota from donor mice. (FIG. 4A-D) Venn diagram based on OTUs similarity between donor (Lean_do and DIO_do) and recipient (Lean_rec and DIO_rec) mice. (FIG. 4E-F) Principal coordinates analysis (PCoA) based on the unweighted UniFrac analysis on operational taxonomic units (OTUs); (FIG. 4E) PCoA PC1 vs PC2; (FIG. 4F) PCoA PC3 vs PC2; [symbol] : Lean_do; ▪: Lean_rec: ●: DIO_do; ▴: DIO_rec.

FIG. 5 is a graph showing the correlations between gut microbes and dopaminergic markers. Spearman's correlation after FDR correction. P-values were obtained after Spearman's correlation test. *: p≤0.05.

FIG. 6A-6B is a set of histograms showing that dopaminergic and opioid systems of gut microbiota recipient mice from obese donors are under-stimulated. Nucleus accumbens mRNA expressions of (FIG. 6A) dopamine receptor 2 (Drd2), dopamine receptor 1 (Drd1), tyrosine hydroxylase (Th), dopamine transporter (Dat), (FIG. 6B) α-opioid receptor (Oprm), κ-opioid receptor (Oprk), 8-opioid receptor (Oprd) and pre-prodynorphin (Pdyn) measured by qPCR in gut microbiota recipient mice from lean (Lean_rec) and diet-induced obese donor mice (DIO_rec). Data are shown as mean±SEM (n=6/group). p-values were obtained after unpaired Student's t-test or non-parametric Mann-Whitney test. *: p-value≤0.05.

FIG. 7A-7B is a set of histograms showing that obese mice show an alteration of the learning component of the food reward, partially transferred by gut microbes. FIG. 7A shows preference score of conditioned place preference based on the difference of time spent(s) in the palatable food-associated side vs the time spent in the neutral-associated side of the cage during the pre-test and the test by lean (Lean_do) or diet-induced obese donor mice (DIO_do). FIG. 7B shows preference score of conditioned place preference based on the difference of time spent(s) in the palatable food-associated side vs the time spent in the neutral-associated side of the cage during the pre-test and the test by gut microbiota recipient mice from lean (Lean_rec) and diet-induced obese donor mice (DIO_rec). Data are shown as mean±SEM (n=6/group). p-values were obtained after paired Student's t-test. *: p-value≤0.05 between preference scores during test and pre-test.

FIG. 8A-8D is a set of graphs showing that gut microbes from obese donors lead to excessive motivation for food reward. Operant conditioning test showing (FIG. 8A) the number of active lever presses and (FIG. 8B) the number of pellets earned during the progressive ratio sessions (PR) by lean (Lean_do) and diet-induced obese donor mice (DIO_do). Operant conditioning test showing (FIG. 8C) the number of active lever presses and (FIG. 8D) the number of pellets earned during the progressive ratio sessions (PR) by gut microbiota recipient mice from lean (Lean_rec) and obese donors (DIO_rec). Data are shown as mean±SEM (n=6/group). p-values were obtained after unpaired Student's t-test. *: p-value≤0.05; **: p-value≤0.01; ***: p-value≤0.001; ****: p-value≤0.0001.

FIG. 9A-9E is a set of histograms showing that homeostatic regulators of food intake are similar between recipient mice. Plasma concentrations of (FIG. 9A) ghrelin, (FIG. 9B) insulin, (FIG. 9C) leptin, (FIG. 9D) Glucagon-like peptide-1 (GLP-1) and (FIG. 9E) Peptide YY (PYY) in gut microbiota recipient mice from lean (Lean_rec) and obese donors (DIO_rec). Data are shown as mean±SEM (n=7-8/group). p-values were obtained after unpaired Student's t-test or non-parametric Mann-Whitney test between lean and obese (DIO) donor and recipient mice separately. **: p-value≤0.01; ***: p-value≤0.001.

FIG. 10 is a histogram showing that Parabacteroides distasonis reduces fat mass gain under HFD. Fat mass of ND PBS, ND PD, HFD PBS, HFD PD after a 8 weeks period. Data are shown as mean±SEM (n=9-10/group). P-values were obtained after One-way ANOVA, followed by Tukey post-hoc test, **: P<0.01; ****: P<0.0001.

FIG. 11 is a histogram showing effect of Parabacteroides distasonis on the liking component of food reward during food preference test. Food preference test showing total HFHS and CT intake after 3 hours of test by ND PBS, ND PD, HFD PBS, HFD PD mice. Data are shown as mean±SEM (n=5-6/group). P-values were obtained after Mann Whitney Test: ** P<0.01 CT vs. HFHS in ND PBS and * P<0.05 CT vs. HFHS ND PD groups.

FIG. 12 is a graph showing that Parabacteroides distasonis reduces motivation to obtain food reward in normal diet-fed mice. Operant wall test showing the number of presses on the active lever to obtain sucrose pellets in ND PBS, ND PD, HFD PBS, HFD PD. Data are shown as mean±SEM (n=6/group). P-values were obtained after Two-way ANOVA repeated measurement, followed by Bonferroni post-hoc test. **** P<0.001 ND PBS vs. HFD PBS; +P<0.05; ++++ P<0.001 ND PBS vs. ND PD.

FIG. 13A-13B is a set of histograms showing effects of Parabacteroides goldsteinii on body weight gain and fat mass under HFD. Body weight gain and (FIG. 13A) and Fat mass (FIG. 13B) of ND PBS, ND PG, HFD PBS, HFD PG after a 5 weeks period. Data are shown as mean±SEM (n=20/group, these data correspond to the results of 2 independent experiments). P-values were obtained after One-way ANOVA, followed by Tukey post-hoc test, *: P<0.05; **: P<0.01; *** P<0.001; **** P<0.0001.

FIG. 14 is a histogram showing effect of Parabacteroides goldsteinii on the liking component of food reward during food preference test. Food preference test showing total HFHS and CT intake after 3 hours session by ND PBS, ND PG, HFD PBS and HFD PG mice. Data are shown as mean±SEM (n=10-12/group, these data correspond to the results of 2 independent experiments). P-values were obtained after Two-way ANOVA, followed by Bonferroni post-hoc test. *: P<0.05; **: P<0.01; P<0.001.

FIG. 15 is a graph showing that Parabacteroides goldsteinii reduces motivation to obtain food reward in normal diet-fed mice. Operant wall test showing the number of presses on the active lever to obtain sucrose pellets in ND PBS, ND PG, HFD PBS, HFD PG mice. Data are shown as mean±SEM (n=11-12/group, these data correspond to the results of 2 independent experiments). P-values were obtained after Two-way ANOVA repeated measurement, followed by Bonferroni post-hoc test. ** P<0.01; *** P<0.001 ND PBS vs. HFD PBS; +P<0.05; +++ P<0.001 ND PBS vs. ND PG; £ P<0.05; ££ P<0.01; £££ P<0.001 ND PBS vs. HFD PG.

FIG. 16 is a histogram showing that Parabacteroides goldsteinii induces a strong positive reinforcement in the learning component of food reward. Conditioned place preference test showing CPP scores in ND PBS, ND PG, HFD PBS, HFD PG mice. Data are shown as mean±SEM (n=11-12/group, these data correspond to the results of 2 independent experiments). P-values were obtained after Two-tailed paired T test: *P<0.05; ** P<0.01; *** P<0.001; **** P<0.0001 pretest vs. test; P-values were obtained after One-way ANOVA, followed by Tukey post-hoc test: $ P<0.05 HFD PG test vs. HFD PBS test.

FIG. 17A-17B is a set of graphs showing that succinate has beneficial effect on obese phenotype. Body weight (FIG. 17A) and Fat mass (FIG. 17B) of ND PBS, ND PD, HFD PBS, HFD PD after a 5 weeks period. Data are shown as mean±SEM (n=10/group). P-values were obtained after Two-way ANOVA repeated measurement, followed by Bonferroni post-hoc test. £ P<0.05; £££ P<0.001 ND vs. HFD; * P<0.05; **: P<0.01; **** P<0.0001 HFD vs. HFD SUC; $ P<0.05 ND vs. ND SUC.

FIG. 18 is a histogram showing that succinate improves the liking component of food reward that is altered during HFD-induced obesity. Food preference test showing total HFHS and CT intake after 3 hours session in ND, HFD, ND SUCC and HFD SUCC mice. Data are shown as mean±SEM (n=5-6/group). P-values were obtained after Two-way ANOVA, followed by Bonferroni post-hoc test. * P<0.05; **: P<0.01; ***: P<0.001; *** P<0.0001 CT vs. HFHS, +P<0.05 HFD SUC HFHS vs. ND SUC HFHS; ++P<0.01 HFD SUC HFHS vs. ND HFHS; ++++P<0.0001, HFD SUC HFHS vs. HFD HFHS.

FIG. 19 is a graph showing that succinate reverses reward system component «wanting» in obesity and reduces motivation to obtain food reward in normal diet-fed mice. Operant wall test showing the number of presses on the active lever to obtain sucrose pellets in ND, HFD, ND SUC, HFD SUC mice. Data are shown as mean±SEM (n=6/group). P-values were obtained after Two-way ANOVA repeated measurement, followed by Bonferroni post-hoc test (£ P<0.05; ££ P<0.01; £££ P<0.001 ND vs. HFD) and unpaired T test for PR separated analysis (*P<0.05 HFD SUC vs HFD; $P<0.05 ND SUC vs. ND).

### EXAMPLES

The present invention is further illustrated by the following examples.

### Example 1

### Materials and Methods

### 1. Mice and Experimental Design

All mouse experiments were approved by the ethical committee for animal care of the Health Sector of the UCLouvain, Université catholique de Louvain under the specific number 2017/UCL/MD/005 and performed in accordance with the guidelines of the local ethics committee and in accordance with the Belgian Law of May 29, 2013 regarding the protection of laboratory animals (agreement number LA1230314).

### 2. Donor Mice

A cohort of 8-week-old specific-opportunistic and pathogen-free (SOPF) male C57BL/6J mice (10 mice, n=5 per group) (Janvier Laboratories®, France) were housed in a controlled environment (room temperature of 22±2° C., 12 h daylight cycle) in groups of two mice per cage, with free access to sterile food (irradiated) and sterile water. Upon delivery, mice underwent an acclimatization period of one week, during which they were fed a control diet (CT, AlN93Mi, Research Diet, New Brunswick, NJ, USA). Then, mice were randomly divided in two groups, and were fed for 5 weeks with control low-fat diet (CT, AlN93Mi) or a high-fat diet (HFD, 60% fat and 20% carbohydrates (kcal/100 g) D12492i, Research diet, New Brunswick, NJ, USA). Body weight, food and water intake were recorded once a week. Body composition was assessed by using 7.5 MHz time domain-nuclear magnetic resonance (TD-NMR, LF50 Minispec, Bruker®, Rheinstetten, Germany). After 4 weeks of follow-up, the mice entered the metabolic chambers to perform the food preference test.

### 3. Recipient Mice

A cohort of 3-week-old specific-opportunistic and pathogen-free (SOPF) male C57BL/6J mice (15 mice, n=7-8 per group) (Janvier Laboratories®, France) were housed in a controlled environment (room temperature of 22±2° C., 12 h daylight cycle) in groups of two mice per cage, with free access to sterile food (irradiated) and sterile water. Mice were fed a low-fat control diet (CT, AlN93Mi) during the entire transplantation protocol as well as after gut microbiota transplantation. Body weight, food and water intake were recorded once a week. Body composition was assessed by using 7.5 MHz time domain-nuclear magnetic resonance (TD-NMR, LF50 Minispec, Bruker®, Rheinstetten, Germany). After 12 weeks of follow-up, the mice entered the metabolic chambers to assess precisely their food intake and metabolism then perform the food preference test.

### 4. Fecal Microbiota Transplantation

At the end of the donor experiment, caecal content was collected in sterile containers and immediately diluted (1:50 w/vol) in sterile Ringer buffer (4.5 g NaCl, 200 mg KCl, 125 mg CaCl₂)). This suspension was then diluted (1:1 v/v) in 20% (w/v) skim milk (Nonfat dry milk, Biorad®, 2005668 A) before storage at −80° C. Two CT-fed mice and two HFD-fed mice from donor cohort were selected as fecal microbiota donors for seven or eight recipient mice per group respectively with 1 donor for 3 or 4 recipient mice. Prior to gut microbiota inoculation, 3-week old SOPF recipient mice were depleted in intestinal microbiota by daily gavage of a broad-spectrum, poorly absorbed mix of antibiotics during 5 days (100 mg/kg of ampicillin, neomycin and metronidazole and 50 mg/kg of vancomycin diluted in sterile water) added with antifungal (amphotericin B 1 mg/kg). Antibiotic treatment was then followed by a bowel cleansing with the administration of 600 μl of PEG solution (PEG/Macrogol 4000, Colofort®, Ipsen, France) by oral gavage in two times at 30 min intervals after a 2-hour fasting. Colonization was then achieved by intragastric gavage with 300 μl of inoculum three times a week for one week. During antibiotics treatment and inoculation, mice were transferred into clean cages 4 times a week. All recipient mice were kept under CT diet (CT, AlN93Mi).

### 5. Metabolic Chambers

After 11 weeks of follow-up, recipient mice were separated and housed individually one week before entering metabolic chambers (Labmaster, TSE systems GmbH, Bad Homburg, Germany). Then they underwent 4 days of metabolic assessment before the food preference test. The mice were analyzed for oxygen consumption, and carbon dioxide production using indirect calorimetry (Labmaster, TSE systems GmbH). These parameters were expressed as a function of whole-body weight. Locomotor activity was recorded using an infrared light beam-based locomotion monitoring system (expressed as beam breaks count per hour). Sensors recorded the precise food intake of each diet every 15 minutes. Inside the chambers, measurements were taken every 15 minutes. The final data representation (total, day or night) corresponds to all the values measured and summed (light phase or dark phase). The means (n=7) were finally compared between groups.

### 6. Food Preference Test

During 3 hours in the daylight, mice were exposed to two kinds of diets: a low-fat, control normal diet (CT, AlN93Mi, Research diet, New Brunswick, NJ, USA) or a high-fat high-sucrose diet (HFHS, 45% fat and 27.8% sucrose (kcal/100 g) D17110301i, Research diet, New Brunswick, NJ, USA) in metabolic chambers (Labmaster/Phenomaster, TSE systems, Germany). Sensors recorded the precise food intake of each diet every 15 minutes.

### 7. Tissue Sampling

At the end of each experiment, mice were fed and exposed for 1 hour to HFHS before anesthesia with isoflurane (Forene®, Abbott, England). This aims to mimic the conditions of the food preference test and stimulate the dopaminergic food reward system. Then the mice were euthanatized by exsanguination and cervical dislocation. Striatum, nucleus accumbens, prefrontal cortex and caudate putamen were precisely dissected, the caecal content was harvested and immediately immersed into liquid nitrogen, then stored at −80° C. for further analysis.

8. RNA Preparation and Real-Time qPCR Analysis

Total RNA was prepared from the striatum using TriPure reagent (Roche®). Quantification and integrity analysis of total RNA was performed by running 2 μl of each sample on an Agilent® 2100 Bioanalyzer (Agilent® RNA 6000 Nano Kit, Agilent). If the RNA integrity number (RIN) obtained less than 6, the sample was excluded from further analyses. cDNA was prepared by reverse transcription of 1 μg total RNA using the GoScript® Reverse Transcriptase kit (Promega®, Madison, WI, USA). Real-time PCR was performed with the QuantStudio 3 real-time PCR system (Thermo Fisher Scientific®, Waltham, MA, USA). Rpl19 RNA was chosen as the housekeeping gene. All samples were performed in duplicate, and data were analyzed according to the 2-ΔΔCT method. The identity and purity of the amplified product were assessed by melting curve analysis at the end of amplification. Sequences of the primers used for real-time qPCR are available in Table 1.

TABLE 1
primers used for real-time qPCR

| SEQ | | |
|---|---|---|
| ID | | |
| NO: | Primer name | Sequence (5′-3′) |
| 1 | RPL19 Forward primer | gaaggtcaaagggaatgtgttca |
| 2 | RPL19 Reverse primer | ccttgtctgccttcagcttgt |
| 3 | Drd2 Forward primer | ccaagaacgtgagggctaag |
| 4 | Drd2 Reverse primer | tgaggatgcgaaaggagaag |
| 5 | Drd1 Forward primer | gagccaacctgaagacacc |
| 6 | Drd1 Reverse primer | tgacagcatctccatttccag |
| 7 | TH Forward primer | gccaaggacaagctcaggaac |
| 8 | TH Reverse primer | atcaatggccagggtgtacg |
| 9 | DAT Forward primer | aaatgctccgtgggaccaatg |
| 10 | DAT Reverse primer | gtctcccgctcttgaacctc |

9. DNA Isolation from Mouse Caecal Samples and Sequencing

Caecal contents were collected and kept frozen at −80° C. until use. Metagenomic DNA was extracted from the caecal content using a QIAamp® DNA Stool Mini Kit (Qiagen®, Hilden, Germany) according to the manufacturer's instructions with modifications (see Everard et al., ISME J 2014; 8:2116-30). The V1-V3 region of the 16S rRNA gene was amplified from the caecal microbiota of the mice using the following universal eubacterial primers: 27Fmod (5′-agrgtttgatcmtggctcag-3′; SEQ ID NO: 11) and 519Rmodbio (5′-gtnttacngcggckgctg-3′; SEQ ID NO: 12). Purified amplicons were sequenced utilizing a MiSeq® following the manufacturer's guidelines. Sequencing was performed at MR DNA (www.mrdnalab.com, Shallowater, TX, USA). Sequences were demultiplexed and processed using the QIIME pipeline (v1.9 using default options: Q25, minimum sequence length=200 bp, maximum sequence length=1,000 bp, maximum number of ambiguous bases=6, maximum number of homopolymers=6, maximum number of primer mismatches=0). For the 22 samples analyzed, 102 OTUs have been identified (97% similarity). The minimum number of sequences per sample was 48,170 and the maximum number of sequences per sample was 86,360. The median number of sequences per sample was 61,143 and the mean number of sequences per sample was 63,7392±10,798 (standard deviation). The Q25 sequence data derived from the sequencing process were analyzed with the QIIME 1.9 pipeline. Briefly, sequences were depleted of barcodes and primers. Sequences 1,000 bp were then removed; sequences with ambiguous base calls and with homopolymer runs exceeding 6 bp were also removed. Sequences were denoised, and operational taxonomic units (OTUs) were generated. Chimeras were also removed. OTUs were defined by clustering at 3% divergence (97% similarity). Final OTUs were taxonomically classified using BLASTn against a curated Greengenes database. PCoA was generated with QIIME using the unweighted UniFrac distance matrix between the samples and as previously described 34, 35 36, 37. Data are available upon request.

### 10. Statistical Analysis

Statistical analyses were performed using GraphPad Prism® version 8.1.2 for Windows (GraphPad® Software, San Diego, CA, USA) except for microbiota analyses as described above. Data are expressed as mean±SEM. Differences between two groups were assessed using unpaired Student's t-test. In case variance differed significantly between groups according to the Fisher test, a non-parametric (Mann-Whitney) test was performed. Differences between more than two groups were assessed using one-way ANOVA or two-way ANOVA if repeated measurements, followed by Tuckey or Bonferroni respectively post-hoc test. In case variance differed significantly between groups, a non-parametric Kruskal-Wallis test was performed, followed by the Dunnett post-hoc test.

### 11. Conditioned Place Preference Test

The learning component of the food reward is evaluated in donor and recipient mice by a Conditioned Place Preference (CPP) test performed in the end of the light phase on a biased apparatus (Phenotyper chambers, Noldus, The Netherlands) as previously described. The behavioral cage is separated in two compartments characterized with smooth or rough floor and black or striped walls. All the compartments were completely cleaned before and after each session. Each session (pre-test, trainings, test) lasts exactly 30 minutes. Locomotor activity is recorded with infrared camera monitoring system and analyzed with the provided software (EthoVision XT 14). On day 1, a pre-test is used to determine the less preferred compartment in baseline (the one in which the mouse spent spontaneously less time) and is defined as the reward-associated compartment (biased CPP method). From day 2 to day 9, donor and recipient mice underwent eight trainings with or without a rewarding stimulus (Reese's®), in the less and in the most preferred compartment respectively (4 sessions in each compartment). During the test, the mouse is free to run in each compartment of the cage (in absence of rewarding stimulus), and the time spent in each compartment is recorded (analyzed with the provided software (Etho Vision XT 14). Preference score is based on the difference of time spent(s) in the palatable food-associated side vs the time spent in the neutral-associated side of the cage during the pre-test and the test.

### 12. Operant Wall Test

The wanting component is linked to the motivation to obtain a reward and is evaluated by an operant wall test in donor and recipient mice as previously described. Each session of the test was conducted during the end of the light phase, in operant conditioning chambers (Phenotyper chambers, Noldus, The Netherlands) and analyzed by the provided software (Ethovision XT 14). Briefly, the mice had intermittent access to an operant wall in their home cages. The operant wall system is composed of two levers and two lights and a pellet dispenser. One lever is arbitrarily designated as active, meaning that pressing on this lever initiates the delivery of a sucrose pellet (5-TUT peanut butter flavoured sucrose pellet, TestDiet, St. Louis, MO) and is associated with a light on. On the other side, another lever associated with a light off, is arbitrarily designated as inactive and will never deliver a reward. Mice were trained for the system twice overnight on a FR schedule (one lever press corresponds to one reward), then underwent 2 sessions of 1 h30. Mice were then shifted to PR sessions (2 h), the number of lever press to obtain a reward is incrementally increased (n+3) for every pellet. Mice that did not press on the active lever during the different sessions have been removed.

### 13. Other Stimuli

Rewarding stimuli distinct from food may be used, such as, e.g., alcohol or drugs.

### Results

### 1. DIO Donor Mice Show Alteration in Hedonic Eating

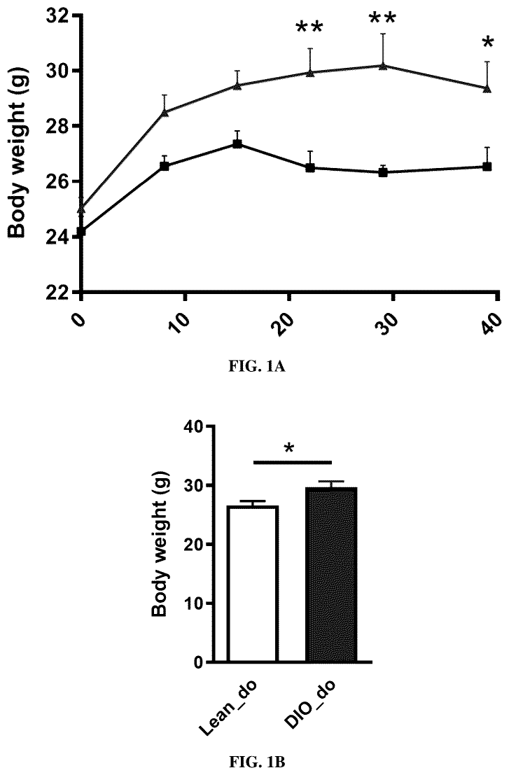

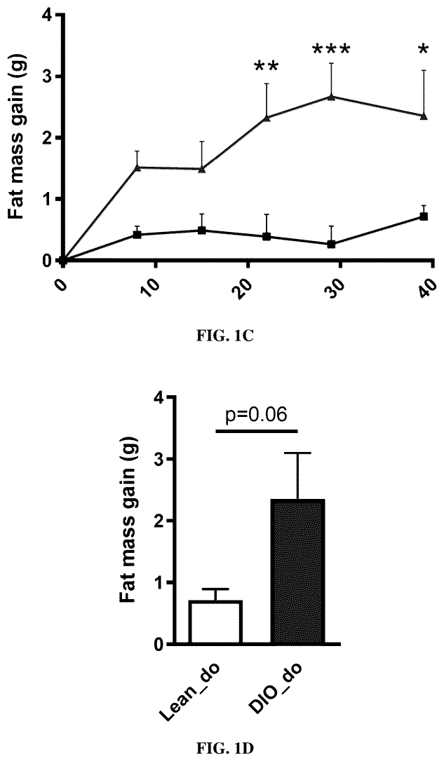

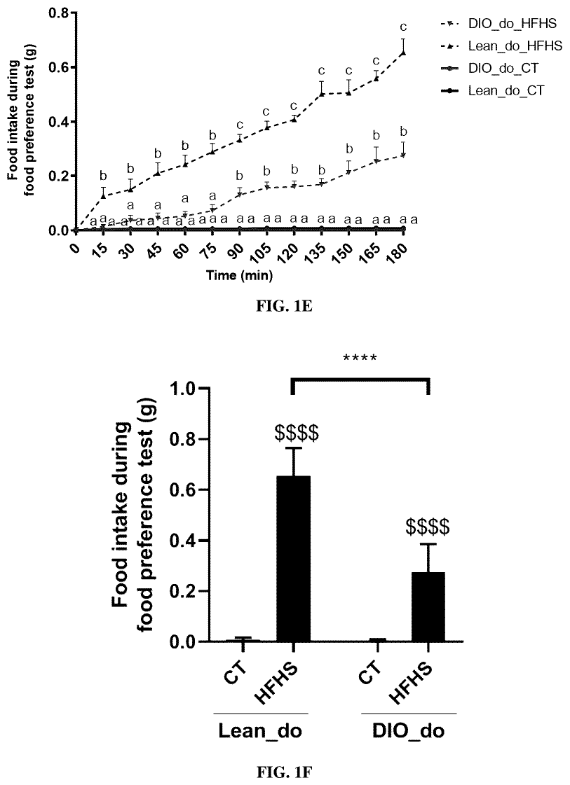

First, 10 donor mice were exposed to low-fat (control, CT) or high-fat diet (HFD) for 5 weeks to induce a lean or obese phenotype (diet-induced obesity, DIO), respectively. As expected, mice fed with an HFD showed an increase of 12% in body weight (FIG. 1A-B) and 230% in fat mass gain (FIG. 1C-D) compared to CT-fed mice. Then, in order to study the hedonic component of food intake, the pleasure associated with palatable food consumption in these mice was analyzed.

To assess spontaneous hedonic food intake, the donor mice underwent a food preference test in which they were exposed for the first time to palatable diet (High-Fat High-Sucrose, HFHS). During this food preference test, donor mice were exposed to HFHS and low-fat control diet (CT) for three hours during the light phase and the consumption of each diet was recorded (FIG. 1E and FIG. 1F). Both lean and obese mice preferred HFHS diet to CT as they ate more HFHS than CT during the food preference test. However, lean mice showed a faster tropism towards HFHS since they ate significantly more HFHS than CT from the beginning of the test, whereas DIO mice preferred significantly palatable diet over control diet only after 90 min (FIG. 1E). Overall, DIO mice were significantly less attracted to palatable diet, eating 58% less HFHS (p<0.0001) than lean mice over the whole food preference test (FIG. 1F).

2. Obese Gut Microbiota Transplantation Transfers Alteration in Hedonic Eating Associated with Obesity

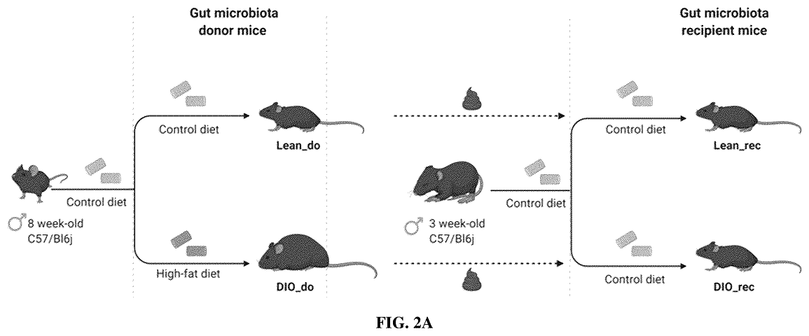

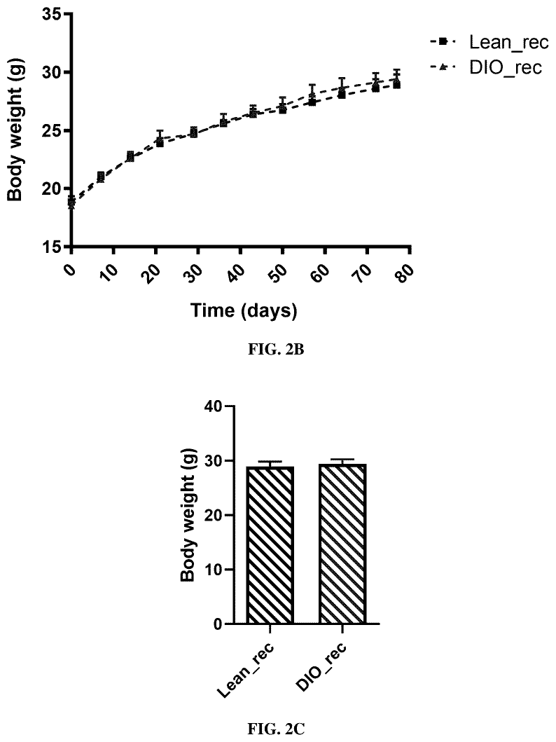

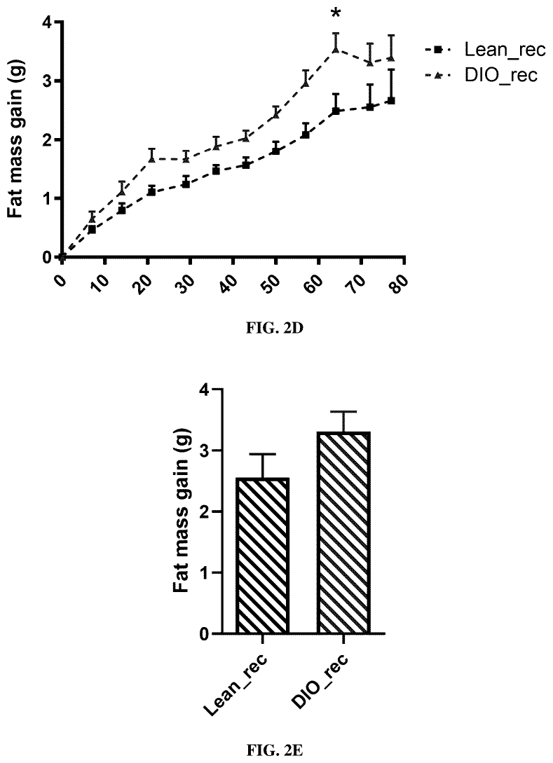

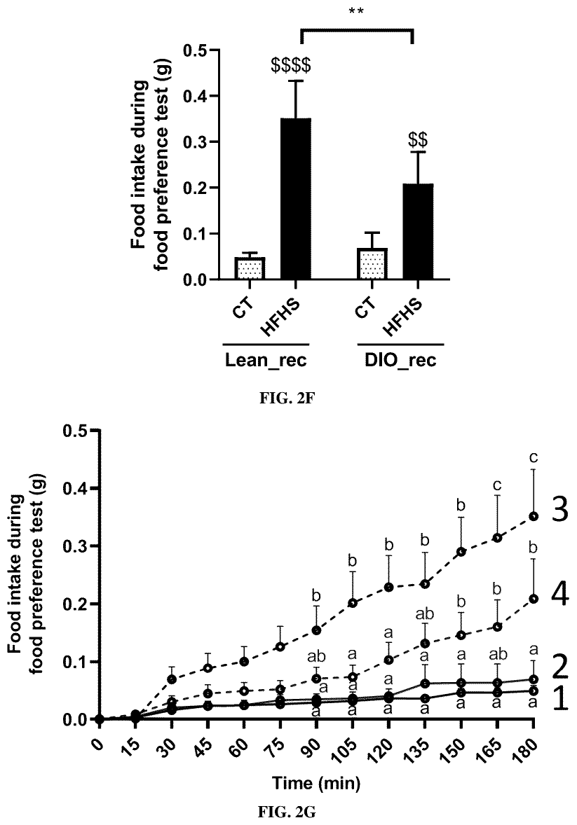

To study the causal role of the gut microbiota in obesity-related hedonic eating disorders, the gut microbiota from 2 lean and 2 obese donor mice were transplanted into 7 and 8 recipient mice respectively. All recipient mice were fed with the same low-fat, control diet during the whole experiment (FIG. 2A).

Lean and obese gut microbiota recipient mice (Lean_rec and DIO_rec, respectively) did not show any difference in terms of body weight (FIG. 2B-C) or fat mass gain (FIG. 2D-E). However, DIO gut microbiota recipient mice tended to gain more fat mass over time, with a statistical significance at day 64 (FIG. 2D). In order to investigate energy metabolism of lean and obese gut microbiota recipient mice, precise measurements of O₂ consumption and CO2 production in metabolic chambers were also performed. It was not observed any differences between mice receiving an obese or lean gut microbiota. These results suggest that donor mice did not transfer their obese phenotype to recipient mice in terms of fat mass and body weight after fecal transplantation.

Interestingly, during the entire follow up, lean and obese gut microbiota recipient mice had similar intake of control diet. However, during their first exposure to palatable food (i.e. food preference test), differences in HFHS intake were revealed (FIG. 2F and FIG. 2G). Lean gut microbiota recipient mice displayed a faster preference for HFHS than DIO gut microbiota recipient mice. In fact, lean gut microbiota recipient mice ate significantly more HFHS than CT diet after 90 minutes of test, whereas the difference between HFHS and CT intake in DIO gut microbiota recipient mice was only significant after 150 and 180 min (FIG. 2G). Like the donor mice, the two recipient groups showed a preference for palatable diet over CT diet. Strikingly, the total HFHS intake was 40% less important in DIO gut microbiota recipient mice compared to lean gut microbiota recipient mice (p<0.01, FIG. 2F). These results demonstrate that lean and DIO gut microbiota recipient mice show similar patterns in terms of hedonic eating behavior as their respective microbiota donors and this effect was independent from obesity development or non-hedonic feeding behavior. Of note, ambulatory activity during the test was comparable between recipient mice, suggesting a similar exploratory behavior towards this novel food high in sugar and fat. Taken together, a causal role of the gut microbiota in the hedonic food behavior alterations associated with obesity was uncovered.

### 3. Dopaminergic Markers in the Striatum Suggest a Hypofunctional Food Reward System in DIO Recipient Mice

Pleasure associated with palatable food intake is mainly driven by dopaminergic pathways in the mesocorticolimbic system. Indeed, ingestion of diet rich in fat and sugar has been shown to be associated with the release of dopamine in the dorsal striatum in proportion to the self-reported level of pleasure derived from eating the food. Dopamine receptors 1 and 2 (DIR and D2R) are the most expressed dopamine receptors of the reward system and the scientific literature describes a downregulation of these receptors in the context of obesity in humans and rodents, which in turn is associated with a reduction of the pleasure related to palatable food ingestion. Since transplantation of obese gut microbiota replicated food preference alterations associated with obesity (FIG. 2F), it was wondered if this was associated with modifications in dopaminergic markers. Therefore, the expression of dopaminergic markers in the striatum of recipient mice was investigated by qPCR.

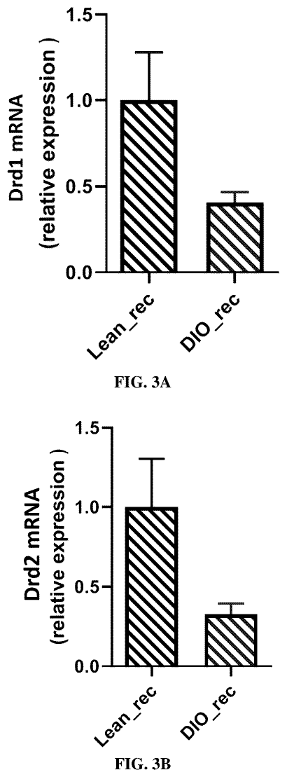

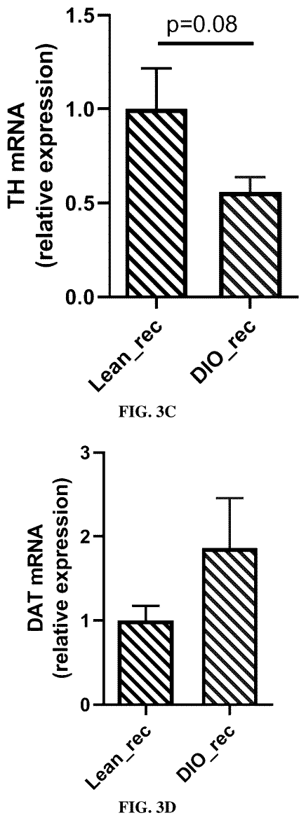

The results show that after microbiota transplantation, DIO recipient mice express at least 60% less Drd1 and Drd2 in the striatum compared to lean recipient mice, although this failed to pass the statistical threshold due to high variability in the Lean_rec group (p>0.05, FIG. 3A-B). The expression of tyrosine hydroxylase (TH), the rate-limiting enzyme synthetizing dopamine, was also decreased (50%) in mice receiving obese microbiota compared to mice receiving lean microbiota (p>0.05, FIG. 3C). In line with these results, the dopamine transporter (DAT), responsible for the recapture of around 80% of the dopamine released, was two-fold more expressed in DIO_rec compared to Lean_rec (p>0.05, FIG. 3D), suggesting low function of the dopaminergic system in obese gut microbiota transplanted mice. Of note, the modifications of expression of dopaminergic markers are not associated with changes in the ambulatory activity, suggesting that the qPCR results observed in the striatum are specific to the reward system rather than the motor function.

Besides the dopaminergic system in the striatum, other brain areas are involved in food reward as caudate putamen, nucleus accumbens and prefrontal cortex. Therefore, it was further investigated and analyzed mRNA levels of the dopaminergic markers in these regions (Table 2).

TABLE 2
mRNA levels of the dopaminergic markers Drd2, Drd1.
TH and DAT in brain areas such as the nucleus accumbens,
the caudate putamen, and the prefrontal cortex.

| | DIO_rec | | | | | | | | |
|---|---|---|---|---|---|---|---|---|---|
| | Nucleus accumbens | | | Prefrontal cortex | | | Caudate putamen | | |
| | | | p- | | | p- | | | p- |
| | Mean | SEM | value | Mean | SEM | value | Mean | SEM | value |
| Drd2 | 1.38 | ±0.64 | 0.60 | 1.22 | ±0.44 | 0.73 | 1.21 | ±0.22 | 0.46 |
| Drd1 | 1.70 | ±0.69 | 0.41 | 1.21 | ±0.27 | 0.56 | 1.15 | ±0.24 | 0.59 |
| TH | 0.97 | ±0.3 | 0.94 | 1.09 | ±0.10 | 0.71 | 0.98 | ±0.07 | 0.85 |
| DAT | | | | 0.59 | ±0.07 | 0.43 | | | |

It was not observed any differences between lean and obese gut microbiota recipient mice in the prefrontal cortex and in the caudate putamen. However, the results tend to show a slight modulation of the expression the dopaminergic markers in the nucleus accumbens.

To confirm these results, another line of experiments was conducted, but this time with the mice maintained in caloric restriction conditions during the test. The expression of dopaminergic and opioid markers in the Nucleus accumbens (NAc) of gut microbiota recipient mice from lean and obese donors was investigated (FIG. 6A-B). A significant decrease in the expression of dopamine receptor 2 (Drd2) and the enzyme synthetizing dopamine, tyrosine hydroxylase (Th) was found in the NAc of mice recipient of gut microbes from obese donors compared to mice recipient of gut microbes from lean donors. Dopamine receptor 1 (Drd1) and the dopamine transporter (Dat) tended to be reduced in mice transplanted with gut microbiota from obese mice compared to mice transplanted with gut microbiota from lean mice (FIG. 6A).

Since the opioid system is also involved in food reward, and has been shown to be blunted in obese conditions, the expression of some key markers was measured and it was found that DIO_rec had a significant reduction in the NAc expressions of u-opioid receptor (Oprm), a similar trend for reduction in k-opioid receptor (Oprk, p=0.05) and the precursor of the dynorphin (Pdyn, pre-prodynorphin, p=0.06, FIG. 6B). The expression of δ-opioid receptor (Oprd) did not differ between Lean_rec and DIO_rec (FIG. 6B).

4. Fecal Material Transplantation from Obese Donors into Lean Recipient Mice is Efficient

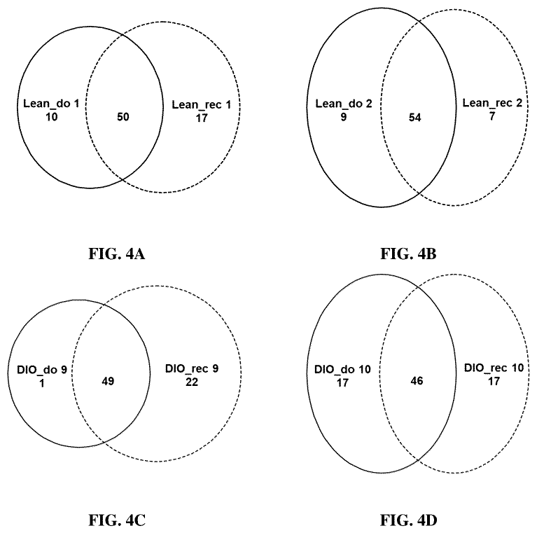

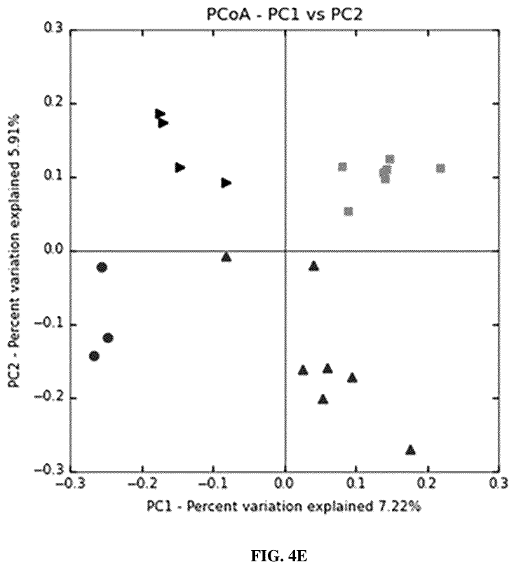

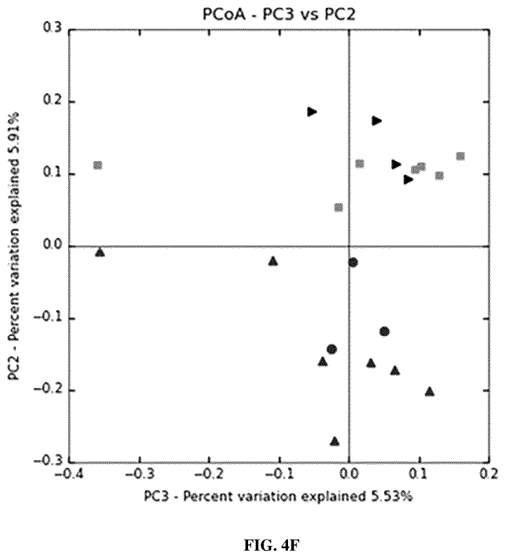

To validate the efficiency of the gut microbiota transplantation, bacterial composition of caecum contents from donor and recipient mice were analyzed using 16S IRNA sequencing. Common OTUs (Operational Taxonomic Units) between donors and recipients were compared at the end of each experiment, just after food preference tests (FIG. 4A-D). Two mice from each donor group (CT-fed or HFD-fed) were donors for 7 Lean_rec and 8 DIO_rec recipient mice respectively with one donor mouse for 3 or 4 recipient mice. Venn diagram showed a high similarity of OTUs (more than 50%) between donors and recipients, confirming the colonization of antibiotic-treated recipients with gut microbiota from donors (FIG. 4A-D).

Furthermore, as represented on the PCoA, obese donors and obese gut recipient mice have gut microbiota profiles that differ from lean donors and lean gut microbiota recipient mice according to the principal component PC2 (FIG. 4E-F).

### 5. Parabacteroides Represents a Potential Link in the Gut-to-Brain Axis Controlling Hedonic Food Intake

As a preliminary approach to highlight a potential link between the gut microbiota and the food reward system in the context of obesity, Spearman's correlations was used to establish associations between several parameters of the food reward system and the gut microbiota. Data from donor and recipient mice were combined to create the correlation matrix. The table showed that 18 OTUs correlated with the total HFHS intake measured during the food preference test (Table 3). In addition, positive correlations were found between an unidentified genus of the Peptococcacede family and mRNA expression of DIR, D2R and TH (Table 3).

TABLE 3
Significant Spearman's correlations between bacterial composition and
food reward patters. Spearman's correlations were calculated for each parameter for
donor and recipient mice. Significant values are highlighted in bold.

| | BW | | BW gain | | Fat mass | | Fat mass gain | |
|---|---|---|---|---|---|---|---|---|
| | Spearman | p | Spearman | p | Spearman | p | Spearman | p |
| | r | value | r | value | r | value | r | value |
| Streptococcaceae; Other | 0.341 | 0.111 | −0.718 | 0.000 | −0.143 | 0.515 | −0.318 | 0.139 |
| Methylobacteriaceae; Other | −0.394 | 0.077 | −0.364 | 0.105 | −0.362 | 0.107 | −0.420 | 0.058 |
| Desulfovibrionaceae; Other | 0.241 | 0.293 | 0.067 | 0.773 | 0.256 | 0.262 | 0.402 | 0.071 |
| Clostridiaceae; Other | −0.013 | 0.957 | −0.342 | 0.129 | 0.006 | 0.981 | −0.240 | 0.295 |
| Bacteria; Other; Other; | −0.050 | 0.831 | 0.127 | 0.582 | 0.176 | 0.445 | 0.340 | 0.131 |
| Other; Other; Other | | | | | | | | |
| S24-7; g_ | −0.201 | 0.381 | 0.031 | 0.893 | −0.114 | 0.622 | −0.157 | 0.498 |
| RF32; f_; g_ | −0.186 | 0.420 | −0.268 | 0.241 | −0.231 | 0.313 | −0.512 | 0.018 |
| Prevotellaceae; g_ | −0.292 | 0.200 | −0.384 | 0.085 | −0.621 | 0.003 | −0.750 | 0.000 |
| Peptostreptococcaceae; g_ | −0.459 | 0.036 | −0.609 | 0.003 | −0.436 | 0.048 | −0.540 | 0.012 |
| Peptococcaceae; g_ | 0.006 | 0.979 | −0.653 | 0.001 | −0.121 | 0.602 | −0.253 | 0.269 |
| Mogibacteriaceae; g_ | −0.392 | 0.079 | −0.490 | 0.024 | −0.506 | 0.019 | −0.461 | 0.036 |
| F16; g_ | −0.433 | 0.050 | −0.671 | 0.001 | −0.453 | 0.039 | −0.558 | 0.009 |
| Desulfovibrionaceae; g_ | −0.046 | 0.843 | −0.279 | 0.220 | 0.021 | 0.929 | 0.088 | 0.703 |
| Coriobacteriaceae; g_ | 0.033 | 0.886 | 0.550 | 0.010 | 0.263 | 0.250 | 0.402 | 0.070 |
| Christensenellaceae; g_ | 0.021 | 0.930 | −0.143 | 0.535 | −0.137 | 0.554 | −0.347 | 0.123 |
| Bacillaceae; g_ | 0.121 | 0.602 | 0.462 | 0.035 | 0.316 | 0.163 | 0.199 | 0.386 |
| Turicibacter | −0.265 | 0.245 | −0.535 | 0.012 | −0.310 | 0.172 | −0.402 | 0.071 |
| Staphylococcus | −0.062 | 0.790 | −0.141 | 0.543 | −0.026 | 0.912 | −0.272 | 0.233 |
| Ruminococcus | 0.120 | 0.606 | 0.311 | 0.170 | 0.220 | 0.339 | 0.194 | 0.399 |
| Prevotella | −0.105 | 0.652 | −0.034 | 0.884 | −0.092 | 0.691 | −0.196 | 0.394 |
| Parabacteroides | −0.213 | 0.354 | −0.283 | 0.214 | −0.325 | 0.151 | −0.486 | 0.026 |
| Lactococcus | 0.316 | 0.163 | −0.138 | 0.552 | 0.127 | 0.582 | −0.153 | 0.509 |
| Knoellia | −0.354 | 0.115 | −0.484 | 0.026 | −0.247 | 0.280 | −0.403 | 0.070 |
| Coprococcus | 0.038 | 0.869 | −0.019 | 0.933 | −0.155 | 0.504 | −0.334 | 0.139 |
| Bacillus | 0.392 | 0.079 | 0.337 | 0.135 | 0.382 | 0.088 | 0.462 | 0.035 |
| Anoxybacillus | −0.071 | 0.759 | 0.073 | 0.754 | −0.010 | 0.964 | 0.190 | 0.409 |
| Allobaculum | −0.108 | 0.642 | 0.405 | 0.068 | 0.019 | 0.933 | 0.136 | 0.557 |
| Akkermansia | 0.099 | 0.670 | −0.003 | 0.991 | 0.014 | 0.951 | −0.120 | 0.604 |
| | Lean mass | | Lean mass gain | | HFHS intake | | D2R qPCR | |
| | Spearman | p | Spearman | p | Spearman | p | Spearman | p |
| | r | value | r | value | r | value | r | value |
| Streptococcaceae; Other | 0.480 | 0.020 | −0.776 | 0.000 | 0.302 | 0.161 | 0.295 | 0.193 |
| Methylobacteriaceae; Other | −0.267 | 0.243 | −0.244 | 0.287 | 0.516 | 0.017 | 0.370 | 0.119 |
| Desulfovibrionaceae; Other | 0.209 | 0.362 | 0.031 | 0.893 | −0.497 | 0.022 | −0.108 | 0.661 |
| Clostridiaceae; Other | −0.036 | 0.875 | −0.290 | 0.203 | 0.634 | 0.002 | 0.015 | 0.951 |
| Bacteria; Other; Other; | −0.281 | 0.218 | 0.082 | 0.724 | −0.548 | 0.010 | −0.215 | 0.377 |
| Other; Other; Other | | | | | | | | |
| S24-7; g_ | −0.297 | 0.190 | −0.137 | 0.554 | −0.258 | 0.259 | −0.447 | 0.055 |
| RF32; f_; g_ | −0.179 | 0.437 | −0.288 | 0.205 | 0.558 | 0.009 | −0.063 | 0.797 |
| Prevotellaceae; g_ | −0.032 | 0.889 | −0.374 | 0.095 | 0.564 | 0.008 | −0.011 | 0.966 |
| Peptostreptococcaceae; g_ | −0.338 | 0.134 | −0.585 | 0.005 | 0.589 | 0.005 | 0.213 | 0.382 |
| Peptococcaceae; g_ | 0.122 | 0.599 | −0.638 | 0.002 | 0.285 | 0.210 | 0.510 | 0.026 |
| Mogibacteriaceae; g_ | −0.243 | 0.289 | −0.427 | 0.053 | −0.033 | 0.887 | −0.114 | 0.642 |
| F16; g_ | −0.199 | 0.387 | −0.680 | 0.001 | 0.523 | 0.015 | 0.357 | 0.134 |
| Desulfovibrionaceae; g_ | −0.055 | 0.814 | −0.234 | 0.308 | 0.339 | 0.133 | 0.516 | 0.024 |
| Coriobacteriaceae; g_ | −0.124 | 0.592 | 0.529 | 0.014 | −0.468 | 0.032 | −0.462 | 0.047 |
| Christensenellaceae; g_ | 0.029 | 0.900 | 0.050 | 0.831 | 0.290 | 0.202 | −0.153 | 0.531 |
| Bacillaceae; g_ | −0.043 | 0.852 | 0.463 | 0.034 | 0.168 | 0.468 | −0.308 | 0.199 |
| Turicibacter | −0.214 | 0.351 | −0.379 | 0.090 | 0.546 | 0.011 | 0.013 | 0.957 |
| Staphylococcus | −0.058 | 0.804 | −0.161 | 0.485 | 0.452 | 0.040 | −0.177 | 0.469 |
| Ruminococcus | 0.006 | 0.978 | 0.200 | 0.384 | −0.514 | 0.017 | −0.228 | 0.348 |
| Prevotella | −0.162 | 0.482 | −0.169 | 0.464 | 0.434 | 0.049 | −0.028 | 0.909 |
| Parabacteroides | −0.079 | 0.733 | −0.133 | 0.565 | 0.857 | 0.000 | 0.188 | 0.442 |
| Lactococcus | 0.427 | 0.053 | 0.003 | 0.991 | 0.427 | 0.054 | 0.284 | 0.238 |
| Knoellia | −0.349 | 0.121 | −0.510 | 0.018 | 0.459 | 0.036 | 0.033 | 0.894 |
| Coprococcus | 0.109 | 0.638 | 0.250 | 0.274 | 0.467 | 0.033 | −0.133 | 0.586 |
| Bacillus | 0.221 | 0.336 | 0.337 | 0.136 | −0.604 | 0.004 | 0.092 | 0.708 |
| Anoxybacillus | −0.168 | 0.467 | 0.116 | 0.617 | −0.454 | 0.039 | 0.002 | 0.993 |
| Allobaculum | −0.188 | 0.414 | 0.518 | 0.016 | −0.345 | 0.126 | −0.432 | 0.065 |
| Akkermansia | 0.182 | 0.430 | −0.192 | 0.404 | −0.056 | 0.810 | 0.126 | 0.606 |
| | D1R qPCR | | TH qPCR | | DAT qPCR | | | |
| | Spearman | p | Spearman | p | Spearman | p | | |
| | r | value | r | value | r | value | | |
| Streptococcaceae; Other | 0.416 | 0.061 | 0.552 | 0.012 | −0.047 | 0.843 | | |
| Methylobacteriaceae; Other | 0.396 | 0.093 | 0.140 | 0.596 | 0.051 | 0.835 | | |
| Desulfovibrionaceae; Other | −0.134 | 0.586 | −0.310 | 0.225 | 0.255 | 0.293 | | |
| Clostridiaceae; Other | 0.046 | 0.850 | −0.108 | 0.677 | −0.186 | 0.446 | | |
| Bacteria; Other; Other; | −0.197 | 0.418 | −0.034 | 0.897 | 0.051 | 0.836 | | |
| Other; Other; Other | | | | | | | | |
| S24-7; g_ | −0.475 | 0.040 | −0.444 | 0.076 | 0.205 | 0.399 | | |
| RF32; f_; g_ | −0.044 | 0.858 | 0.091 | 0.730 | −0.147 | 0.547 | | |
| Prevotellaceae; g_ | 0.009 | 0.972 | 0.140 | 0.592 | −0.181 | 0.459 | | |
| Peptostreptococcaceae; g_ | 0.226 | 0.351 | 0.105 | 0.691 | 0.030 | 0.902 | | |
| Peptococcaceae; g_ | 0.555 | 0.014 | 0.510 | 0.039 | 0.065 | 0.792 | | |
| Mogibacteriaceae; g_ | −0.053 | 0.831 | 0.186 | 0.473 | 0.574 | 0.010 | | |
| F16; g_ | 0.349 | 0.143 | 0.356 | 0.167 | 0.130 | 0.595 | | |
| Desulfovibrionaceae; g_ | 0.605 | 0.006 | 0.306 | 0.231 | −0.165 | 0.500 | | |
| Coriobacteriaceae; g_ | −0.490 | 0.033 | −0.431 | 0.085 | 0.320 | 0.182 | | |
| Christensenellaceae; g_ | −0.177 | 0.468 | 0.033 | 0.900 | −0.531 | 0.019 | | |
| Bacillaceae; g_ | −0.319 | 0.183 | −0.014 | 0.960 | −0.469 | 0.043 | | |
| Turicibacter | 0.054 | 0.828 | −0.178 | 0.492 | 0.107 | 0.663 | | |
| Staphylococcus | −0.116 | 0.636 | −0.022 | 0.935 | −0.320 | 0.181 | | |
| Ruminococcus | −0.225 | 0.355 | −0.125 | 0.632 | 0.004 | 0.989 | | |
| Prevotella | 0.016 | 0.949 | 0.007 | 0.981 | −0.400 | 0.090 | | |
| Parabacteroides | 0.147 | 0.547 | 0.152 | 0.559 | −0.351 | 0.141 | | |
| Lactococcus | 0.260 | 0.283 | 0.493 | 0.047 | −0.268 | 0.267 | | |
| Knoellia | 0.072 | 0.768 | 0.105 | 0.691 | −0.076 | 0.758 | | |
| Coprococcus | −0.175 | 0.473 | −0.086 | 0.744 | −0.454 | 0.051 | | |
| Bacillus | 0.103 | 0.676 | 0.002 | 0.994 | −0.292 | 0.225 | | |
| Anoxybacillus | −0.017 | 0.945 | 0.003 | 0.996 | −0.179 | 0.464 | | |
| Allobaculum | −0.537 | 0.018 | −0.429 | 0.087 | 0.096 | 0.694 | | |
| Akkermansia | 0.156 | 0.523 | 0.517 | 0.036 | 0.111 | 0.652 | | |

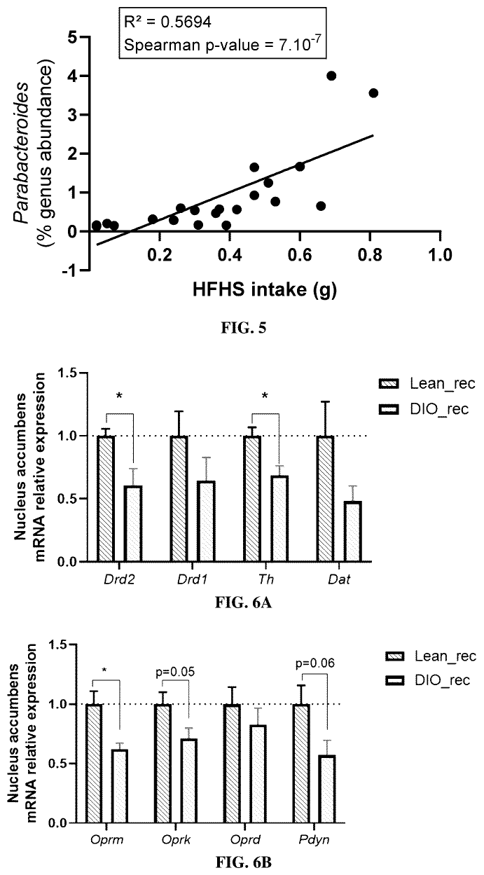

However, after correcting for multiple comparisons using the FDR (false discovery rate) method, only Parabacteroides remained highly positively correlated with the HFHS intake (FIG. 5). This suggested that the more Parabacteroides the mice had, the more HFHS they ate during the food preference test and this behavior implies a functional reward system.

6. Fecal Material Transplantation from Obese Donors Alters the Learning Component

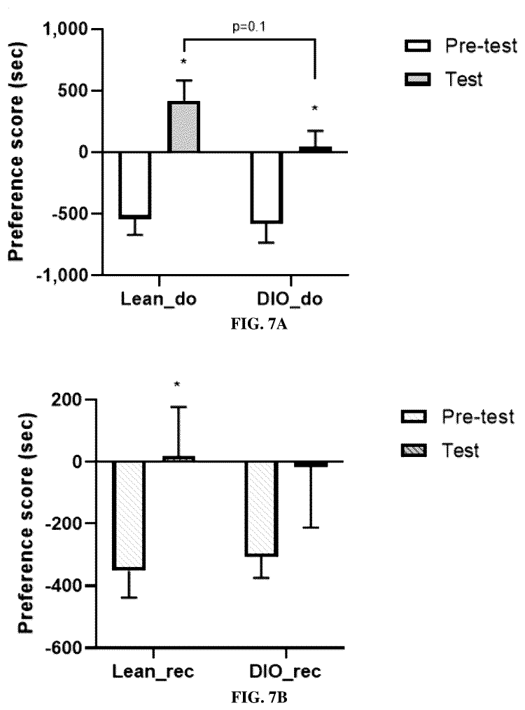

To investigate the roles of gut microbes in the learning, the learning component of the food reward was assessed by CPP test in donor and recipient mice (FIG. 7A-B). The aim of this test is to evaluate to what extent mice could be conditioned to prefer a compartment with a food stimulus, even after the stimulus was removed. Here the goal was to increase the time spent by the mouse in one side of the cage after being restrained in this side during the training sessions with a palatable food pellet stimulating the reward system (Reese's®). A pre-test is used to determine whether mice had a pre-existing preference for any of the compartments at baseline.

Both lean and obese donors spent more time in the compartment associated with palatable food during the test than during the pre-test, suggesting that they are both able to reverse their initial preference for one side of the cage after the training sessions (FIG. 7A). However, the learning component of the food reward is more efficient in lean mice than in obese mice. Indeed, the difference of time spent in the palatable food-associated compartment compared to neutral compartment tends to be lower in obese mice compared to lean mice (p=0.1, FIG. 7A).

Recipients of gut microbiota from lean donors also reversed their initial preference for one compartment and significantly increased the time spent in the palatable side during the test as compared to the pre-test (FIG. 7B). In opposition, even if they spent more time in the palatable associated compartment during the test, gut microbiota recipient mice from obese donors showed no significant difference in the preference score for the palatable side during the test as compared to the pre-test (FIG. 7B). The DIO_rec group failed to reverse their initial preference for one side of the cage, reflecting their inability to effectively associate the side of the cage with palatable food-induced pleasure. These results suggest that recipient mice of gut microbiota from obese donors have a dysregulated learning component of the food reward. Altogether, these data demonstrate that the alterations of the learning component associated with obesity are partially transferred through FMT between donor and recipient mice.

7. Gut Microbiota Recipient Mice from Obese Donors Show Excessive Motivation for Food Reward

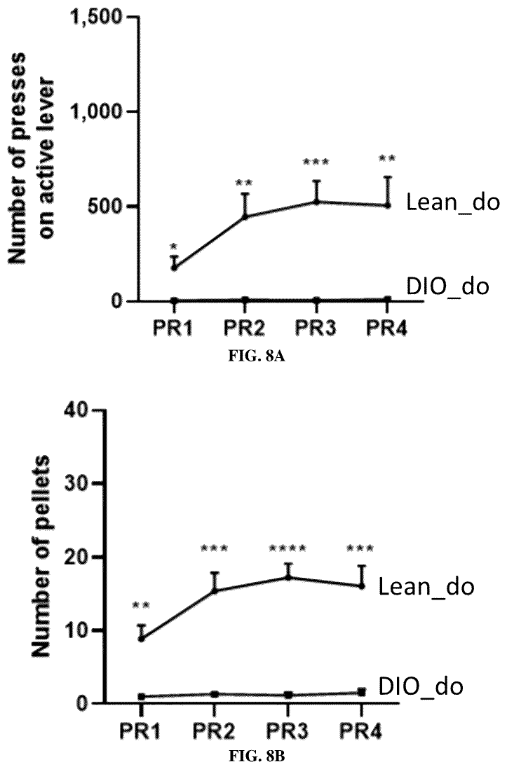

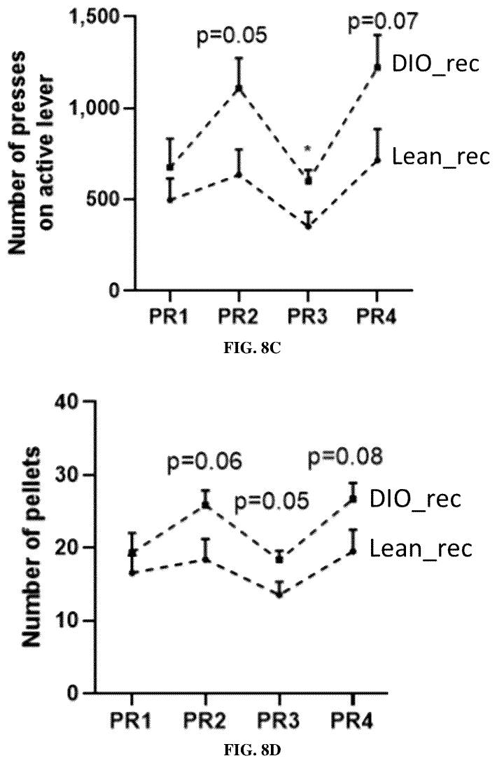

To assess the wanting component or the motivation to obtain food reward, donor and recipient mice underwent an operant wall test in which they had to press on a lever to receive a rewarding sucrose pellet (FIG. 8A-D). The first three sessions of the test were based on a fixed ratio (FR) principle: one food reward required one lever press. Then, in progressive ratios sessions (PR), mice had to press an incrementally increasing number of times (n+3) on the lever in order to obtain each new sucrose pellet, to assess their motivation to obtain a food reward.

Obese mice pressed significantly less on the lever during PR sessions as compared to lean mice (FIG. 8A). The number of reward pellets obtained is also significantly lower for obese than for lean donors during PR sessions (FIG. 8B). As PR sessions are the better reflection of the motivation to obtain a reward, our data show that obesity is associated with an alteration of the wanting component of the food reward.

Surprisingly, gut microbiota recipient mice from obese donors pressed more on the lever during PR sessions 2, 3 and 4 (p=0.05 during PR2, p<0.05 during PR3, p=0.07 during PR4), as compared to lean gut microbiota recipient mice (FIG. 8C). This tendency was reflected by the higher number of rewards obtained by the mice inoculated with gut microbes from obese donors during the PR sessions 2, 3 and 4 (FIG. 8D). These results suggest that gut microbiota recipient mice from obese donors behave in an opposite way as their obese donors in this test assessing the motivation to obtain a reward, since the former pressed approximately 100 times more on the lever to obtain a food reward than the latter. It is worth noting that the absolute values of number of lever presses are similar between Lean_rec and Lean_do groups (FIG. 8A, 8C). Gut microbiota recipient mice from obese donors showed particularly higher values of active lever presses (FIG. 8D), suggesting an excessive motivation for a food reward, rather than a normal motived behavior observed in lean conditions.

8. Excessive Motivation for Food Reward is not Associated with Modulations of Homeostatic Regulators of Food Intake

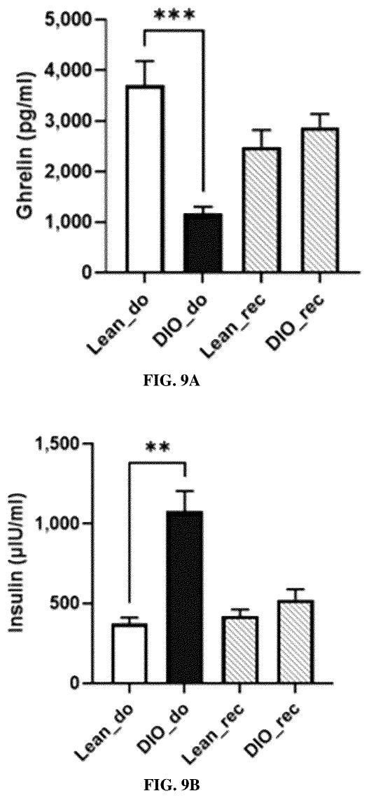

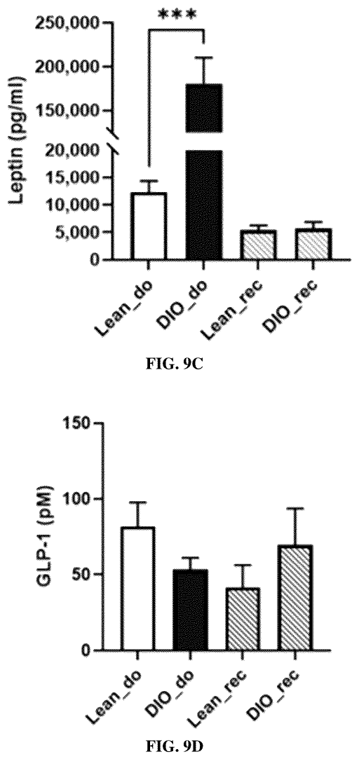

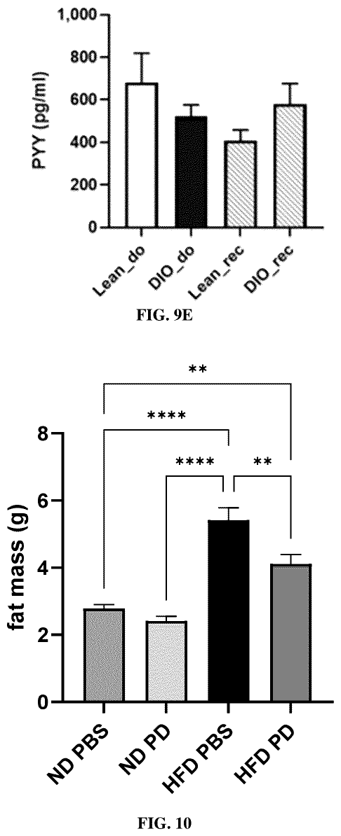

To understand how gut microbes from obese mice could act on the behavioral and neuronal reward system in lean conditions (recipient mice), several mediators of the gut-brain axis involved in the regulation of homeostatic food intake were analyzed, that are also able to influence the food reward system. Therefore, ghrelin, insulin, leptin, GLP-1 and PYY was measured in the plasma of recipient mice, as well as in donor mice. None of the homeostatic regulators analyzed in the plasma were different between gut microbiota recipient mice from lean and obese donors (FIG. 9A-E). In contrast, typical hormonal changes associated with obesity were observed in the plasma of obese donor mice such as a significant decrease in ghrelin (FIG. 9A), a significant increase in insulinemia (FIG. 9B) and leptinemia (FIG. 9C) compared to lean mice. Plasma levels of GLP-1 and PYY were not significantly different between lean and obese donor mice (FIG. 9D-E).

### Example 2

### Materials and Methods

### 1. Mice and Experimental Design

All mouse experiments were approved by the ethical committee for animal care of the Health Sector of the UCLouvain, Université catholique de Louvain under the specific number 2021/UCL/MD/061 and performed in accordance with the guidelines of the local ethics committee and in accordance with the Belgian Law of May 29, 2013 regarding the protection of laboratory animals (agreement number LA1230314).

A cohort of 9-week-old specific-opportunistic and pathogen-free (SOPF) male C57BL/6J mice (Janvier laboratories, Le Genest-Saint-Isle, France) were housed in a controlled environment (room temperature of 22±2° C., 12 h daylight cycle) in groups of two mice per cage, with free access to sterile food (irradiated) and sterile water. Upon delivery, mice were allowed to acclimatize during one week, during which they were fed a control low-fat diet (ND, AlN93Mi, Research Diet, New Brunswick, NJ, USA). Mice were then randomly divided in four groups (40 mice, n=10/group named ND PBS, ND PD, HFD PBS, HFD PD), and fed for 8 weeks with control low-fat diet (ND, AlN93Mi) or a high-fat diet (HFD), 60% fat and 20% carbohydrates (kcal/100 g) (D12492i, Research diet, New Brunswick, NJ, USA). Daily treatment by oral administration with 2×10⁸ Colony-forming unit (CFU) of Parabacteroides distasonis (PD) per mouse in 200 μL of anaerobic PBS containing 1.2% glycerol were conducted on ND PD and HFD PD groups. Daily treatment by oral administration of an equivalent volume of sterile PBS containing 1.2% glycerol were conducted on ND and HFD control groups. Body weight was recorded once a day. Body composition was assessed weekly by using 7.5 MHz time domain-nuclear magnetic resonance (TD-NMR, LF50 Minispec, Bruker, Rheinstetten, Germany). After 4 weeks of follow-up, the mice were placed in behavioral cages (Phenotyper, Noldus, Wageningen, The Netherlands) to perform the food preference test and the operant wall test. During the last test, mice were food-restricted and body weights were maintained at 85% of the initial body weight (before the behavioral tests), as previously described. The caloric restriction allowed to potentiate the reward response to the stimulus.

2. Parabacteroides distasonis Cultivation and Preparation

Parabacteroides distasonis was cultivated on anaerobic liquid YCFA medium and agar YCFA medium. Parabacteroides distasonis was collected by centrifugation (4000 g during 20 minutes twice at 4° C.) and resuspended in sterile PBS with 25% glycerol then immediately frozen in anaerobic vials and stored at −80° C. Before administration, cell pellets were resuspended in anaerobic PBS.

### 3. Food Preference Test

During 3 hours in the daylight, mice were exposed to two diets: a low-fat, control diet (CT, AlN93Mi, Research diet, New Brunswick, NJ, USA) or a high-fat high-sucrose diet (HFHS, 45% fat and 27.8% sucrose (kcal/100 g) D17110301i, Research diet, New Brunswick, NJ, USA) in behavioral cages (Phenotyper, Noldus, Wageningen, The Netherlands). The food intakes were recorded during a 3-hour session in the end of the light phase, in satiated state (access to food ad libitum before and after the test). Mice showing an important spillage of food during the test have been removed.

### 4. Operant Wall Test

The wanting component is linked to the motivation to obtain a reward and is evaluated by an operant wall test as previously described, with some adaptations. Each session of the test was conducted during the end of the light phase, in operant conditioning chambers (Phenotyper, Noldus, The Netherlands) and analyzed by the provided software (Ethovision XT 14). The mice had intermittent access to an operant wall in their home cages. The operant wall system is composed of two levers and two lights and a pellet dispenser. One lever is arbitrarily designated as active, meaning that pressing on this lever initiates the delivery of a sucrose pellet (5-TUT peanut butter flavoured sucrose pellet, TestDiet, St. Louis, MO, USA) and is associated with a light on. On the other side, another lever associated with a light off, is arbitrarily designated as inactive and will never deliver a reward. Mice were trained for the system twice overnight on a fixed-ratio schedule (one lever press on the active lever press corresponds to one reward), then underwent 4 sessions of 1 h30. Mice were then shifted to progressive ratio sessions (PR) (2 h). During the PR sessions, the number of lever presses on the active lever to obtain a reward is incrementally increased (n+3) for every pellet. Mice that did not press on the active lever during the different sessions have been removed.

### 5. Statistical Analysis

Statistical analyses were performed using GraphPad Prism version 9.1.2 for Windows (GraphPad Software, San Diego, CA, USA). Data are expressed as mean±SEM. Differences between groups were assessed using One-way ANOVA, followed by Tukey post-hoc tests. Differences between groups and different time points were assessed using a two-way ANOVA repeated measurement, followed by Bonferroni post-hoc test. Outliers have been excluded after Grubbs test.

### 6. Other Stimuli

Rewarding stimuli distinct from food may be used, such as, e.g., alcohol or drugs.

### Results

1. Effects of Parabacteroides distasonis on Fat Mass Gain

To assess the effects of Parabacteroides distasonis on fat mass, mice were exposed to ND and HFD for 8 weeks and a daily administration of Parabacteroides distasonis or vehicle (PBS) was conducted in ND PD/HFD PD and ND PBS/HFD PBS groups respectively (FIG. 10). As expected, mice fed with HFD showed a significant increase in fat mass over time compared to ND mice. Furthermore, a significant decrease in fat mass is also observed in HFD PD mice as compared to HFD PBS (P<0.05).

2. Effects of Parabacteroides distasonis on the Liking Component of Food Reward

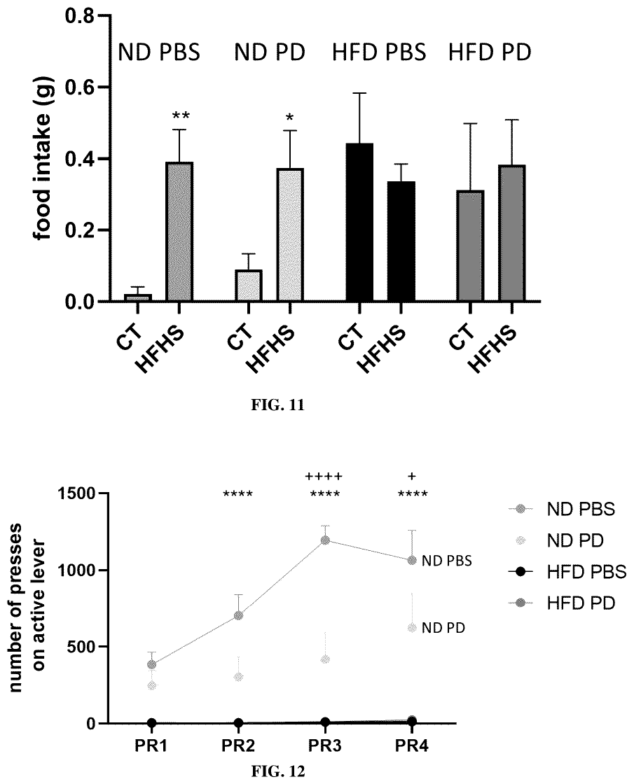

As part of the study of hedonic food intake, a food preference test was performed at the fourth week of exposure to the different diets (ND and HFD). During this test, mice are exposed to a control diet (CT) as well as to a new food that is palatable (HFHS), thus allowing to assess the “liking” component of the food reward system. The consumption of the different foods was measured (FIG. 11). When comparing the amounts of control (CT) and palatable foods (HFHS) consumed during the test, ND PBS mice consumed significantly more HFHS than CT (p<0.01 according to Mann-Whitney test) but no significant differences were observed in HFD PBS groups.

These results demonstrate the impairment of liking component of food intake during HFD-induced obesity. No significant differences in palatable and control food consumption were observed between HFD PBS and HFD PD mice.

These results suggest that Parabacteroides distasonis does not impact the liking component of the reward system in either lean or obese contexts.

3. Effects of Parabacteroides distasonis on Motivation to Obtain Food Reward

In order to further characterize the different components of the food reward system and in particular the motivation of the mice to obtain a food reward, (i.e., the “wanting” component of food intake), an operant wall test was performed and the motivation of the mice was assessed during the progressive ratios sessions (FIG. 12). This test showed a significant decrease in the number of presses on the active lever to obtain sucrose pellets of HFD PBS mice compared to ND PBS mice during PR1, PR2 and PR3 sessions, reflecting a deficit in behaviors associated with the wanting component of the reward system during obesity. No significant differences were observed in the number of active lever presses between the HFD PBS and HFD PD groups.

Surprisingly, mice receiving Parabacteroides distasonis under ND pressed significantly fewer times on the active lever compared to ND PBS mice during PR3 (P<0.0001) and PR4 sessions (P<0.05). Since in control condition, under normal diet, a reduction of active lever press has been associated with a reduction of binge-type eating, these results reveal a potential beneficial effect of Parabacteroides distasonis in the control of wanting a food reward, in lean context.

These results support the use of Parabacteroides distasonis for treating eating-related disorders, and more generally reward for treating dysregulation disorders wherein the wanting component is over-stimulated, typically in patients having compulsive behavior towards a rewarding stimulus.

### Example 3

### Materials and Methods

### 1. Mice and Experimental Design

### See Example 2.

2. Parabacteroides goldsteinii Cultivation and Preparation

Parabacteroides goldsteinii (19448) was purchased from the Deutsche Sammlung von Mikroorganismen und Zellkulturen (DSMZ, Germany). Parabacteroides goldsteinii was cultivated on anaerobic liquid YCFA medium and agar YCFA medium. Parabacteroides goldsteinii was collected by centrifugation (4000 g during 20 minutes, twice, at 4° C.) and resuspended in sterile PBS with 25% glycerol then immediately frozen in anaerobic vials and stored at −80° C. Before administration, cell pellets were resuspended in anaerobic PBS.

### 3. Food Preference Test and Operant Wall Test

### See Example 2.

### 5. Conditioned Place Preference Test

### See Example 1.

### 6. Statistical Analysis

Statistical analyses were performed using GraphPad Prism version 9.1.2 for Windows (GraphPad Software, San Diego, CA, USA). Data are expressed as mean±SEM. Differences between CPP score during the pre-test and the test were assessed using paired Student's t-test. Differences between groups were assessed using a One-way ANOVA, followed by Tuckey post-hoc test. Differences between groups and different time points were assessed using a two-way ANOVA repeated measurement, followed by Bonferroni post-hoc test. Outliers have been excluded after Grubbs test.

### Results

1. Effects of Parabacteroides goldsteinii on Body Weight Gain and Fat Mass

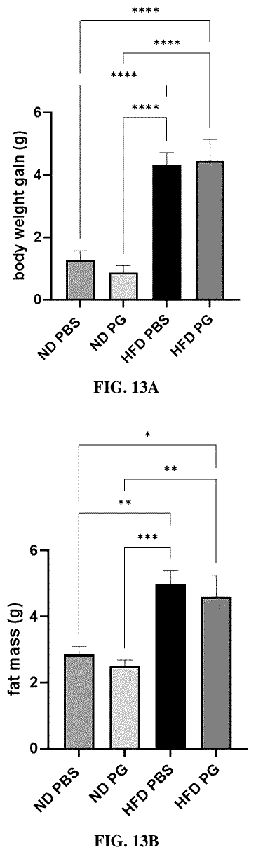

To assess the effects of Parabacteroides goldsteinii on obese phenotype, mice were exposed to ND and HFD for five weeks and a daily administration of Parabacteroides goldsteinii or vehicle (PBS) was conducted in ND PG/HFD PG and ND PBS/HFD PBS groups respectively (FIG. 13A-13B). As expected, mice fed a HFD show a significant increase in body weight gain (P<0.0001) and fat mass (P<0.01) compared to ND-fed mice. However, no significant difference was observed in mice receiving daily administration of Parabacteroides goldsteinii as compared to placebo. These results suggest that Parabacteroides goldsteinii has no significant effect on the obese phenotype induced by a HFD. It should also be noted that no significant difference was observed between the ND PBS and ND PG groups regarding body weight gain and fat mass, suggesting no effect of Parabacteroides goldsteinii in ND fed condition on these parameters.

2. Effects of Parabacteroides goldsteinii on the Liking Component of Food Reward

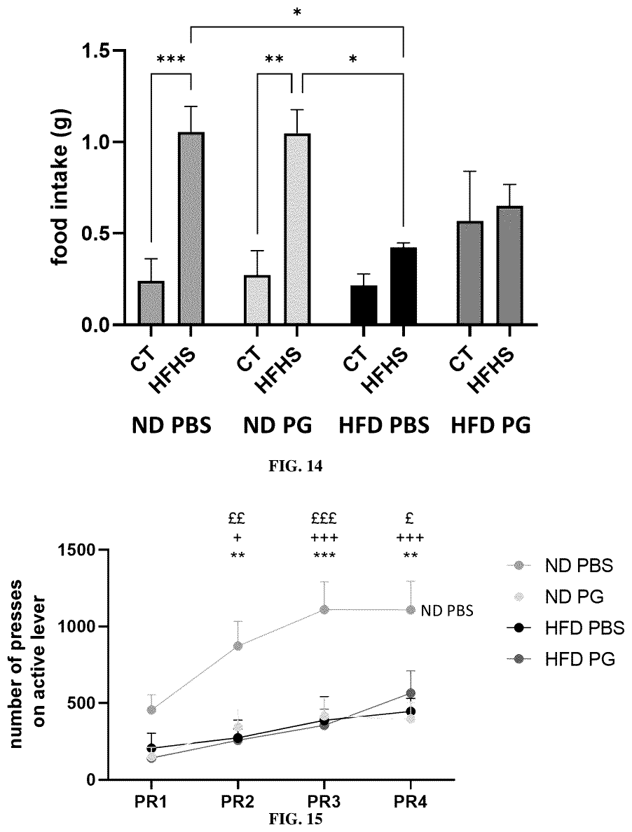

In order to evaluate the pleasure associated with food intake, a food preference test was performed after 5 weeks of exposure to the different diets (ND and HFD). During this test, mice are exposed to a control diet (CT) as well as to a new food that is palatable (HFHS), thus allowing to assess the “liking” component of the food reward system. The consumption of the different foods was measured (FIG. 14). When comparing the amounts of CT and HFHS food eaten during this test, mice in ND PBS and ND PG groups eat significantly more palatable (HFHS) food than control food (CT) (P<0.001; P<0.01). However, no significant difference was observed in terms of HFHS ingested between ND PBS and ND PG mice. In obese conditions, HFD PBS and HFD PG mice do not show any significant difference between palatable food (HFHS) and control food (CT) intake. Furthermore, HFD PBS consumed significantly less palatable food than ND PBS (P<0.05).

These results suggest that Parabacteroides goldsteinii does not impact the liking component of the reward system in either lean or obese contexts.
3. Effects of Parabacteroides goldsteinii on the Motivation to Obtain Food Reward

In order to further characterize the different components of the food reward system and in particular the motivation of the mice to obtain a food reward, (i.e., the “wanting” component of food intake), an operant wall test was performed and the motivation of the mice was assessed during the progressive ratio sessions (FIG. 15). This test showed a significant decrease in the number of presses on the active lever to obtain sucrose pellets of HFD PBS mice compared to ND PBS mice during PR2 (P<0.01), PR3 (P<0.001) and PR4 sessions (P<0.01), reflecting a deficit in behaviors associated with the wanting component of the reward system during obesity. No significant differences were observed in the number of active lever presses between the HFD PBS and HFD PG groups.

Surprisingly, mice receiving Parabacteroides goldsteinii under ND also pressed significantly fewer times on the active lever compared to ND PBS mice during PR2 (P<0.05), PR3 (P<0.001) and PR4 (P<0.001) sessions. Since in control condition, under normal diet, a reduction of active lever press has been associated with a reduction of binge-type eating, these results highlight a potential beneficial effect of Parabacteroides goldsteinii in the control of wanting in the food reward system, in lean context.

These results support the use of Parabacteroides goldsteinii for treating eating-related disorders, and more generally reward for treating dysregulation disorders wherein the wanting component is over-stimulated, typically in patients having compulsive behavior towards a rewarding stimulus.

4. Effects of Parabacteroides goldsteinii on Positive Reinforcement in the Learning Component of Food Reward

To explore another component of the food reward system, the “learning”, a conditioned place preference test was used. The aim of this test is to evaluate to what extent mice could be conditioned to prefer a compartment with a food stimulus, even after the stimulus was removed. The goal was to increase the time spent by the mouse in one side of the cage after being restrained in this side during the training sessions with a palatable food pellet stimulating the reward system (Reese's®). A pre-test is used to determine whether mice had a pre-existing preference for any of the compartments at baseline.

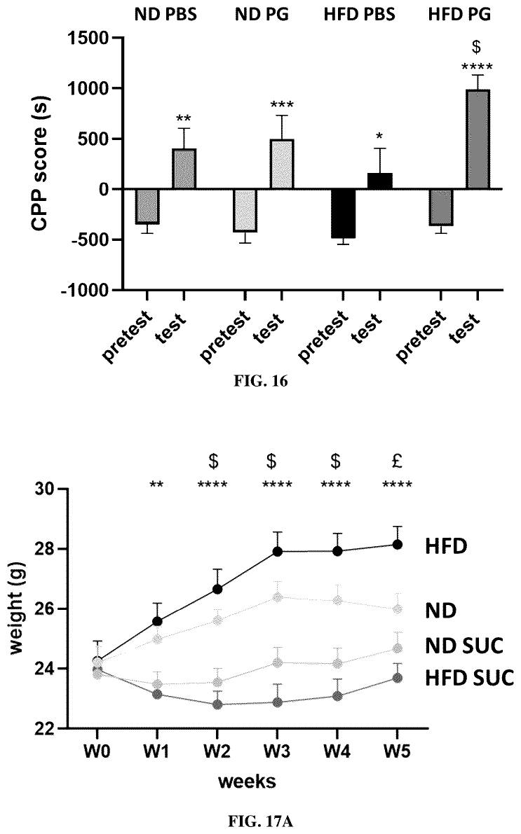

As shown on FIG. 16, regarding the time spent in the compartment associated with palatable food (Reese's®), conditioning sessions induced a significant increase of time spent in this compartment during test compared with the time spent during pretest in ND PBS mice (P<0.01). This effect was also observed in HFD PBS mice but with a lower significant effect (P<0.05). For ND PG mice, a positive reinforcement was also observed by a significant increase of time spent in compartment during test compared to the time spent in the compartment during pretest (P<0.001).

Interestingly, the administration of Parabacteroides goldsteinii in the HFD PG group induced strong positive reinforcement reflected by a significant increase of the time spent in the compartment during the test compared to the time spent in the compartment during the pretest (P<0.0001). Additionally, during the test, the HFD PG mice show significantly higher CPP scores than the HFD PBS CPP scores (P<0.05).

These results support a diet-dependent effect of Parabacteroides goldsteinii on the learning component of the reward system associated with food and any other stimulus associated with reward system.

### Example 4

### Materials and Methods

### 1. Mice and Experimental Design

All mouse experiments were approved by the ethical committee for animal care of the Health Sector of the UCLouvain, Université catholique de Louvain under the specific number 2017/UCL/MD/005 and performed in accordance with the guidelines of the local ethics committee and in accordance with the Belgian Law of May 29, 2013 regarding the protection of laboratory animals (agreement number LA1230314).

A cohort of 9-week-old specific-opportunistic and pathogen-free (SOPF) male C57BL/6J mice (Janvier laboratories, Le Genest-Saint-Isle, France) were housed in a controlled environment (room temperature of 22±2° C., 12 h daylight cycle) in groups of two mice per cage, with free access to sterile food (irradiated) and sterile water. Upon delivery, mice were allowed to acclimatize during one week, during which they were fed a control low-fat diet (ctrl, AlN93Mi, Research Diet, New Brunswick, NJ, USA). Mice were then randomly divided in four groups (40 mice, n=10/group named ND, HFD, ND SUCC, HFD SUCC), and fed for 8 weeks with control low-fat diet (ND), 10 kcal % fat (D1245Oji, Research Diet, New Brunswick, NJ, USA), a high-fat diet (HFD), 60 kcal % fat (D12492i, Research Diet, New Brunswick, NJ, USA), ND supplemented with sodium succinate (W327700, Sigma) at 5% w/w and HFD supplemented with sodium succinate at 5% w/w. Sodium level were matched across all diets. Body weight was recorded weekly. Body composition was assessed weekly by using 7.5 MHz time domain-nuclear magnetic resonance (TD-NMR, LF50 Minispec, Bruker, Rheinstetten, Germany). After 4 weeks of follow-up, the mice were placed in behavioral cages (Phenotyper, Noldus, Wageningen, The Netherlands) to perform the food preference test and the operant wall test. During this last test, mice were food-restricted and body weights were maintained at 85% of the initial body weight (before the behavioral tests), as previously described. The caloric restriction allowed to potentiate the reward response to the stimulus.

### 2. Food Preference Test

### See Example 2.

### 3. Operant Wall Test

### See Example 2.

### 4. Statistical Analysis

### See Example 2.

### 5. Other Stimuli

Rewarding stimuli distinct from food may be used, such as, e.g., alcohol or drugs.

### Results

### 1. Effects of Succinate on Obese Phenotype

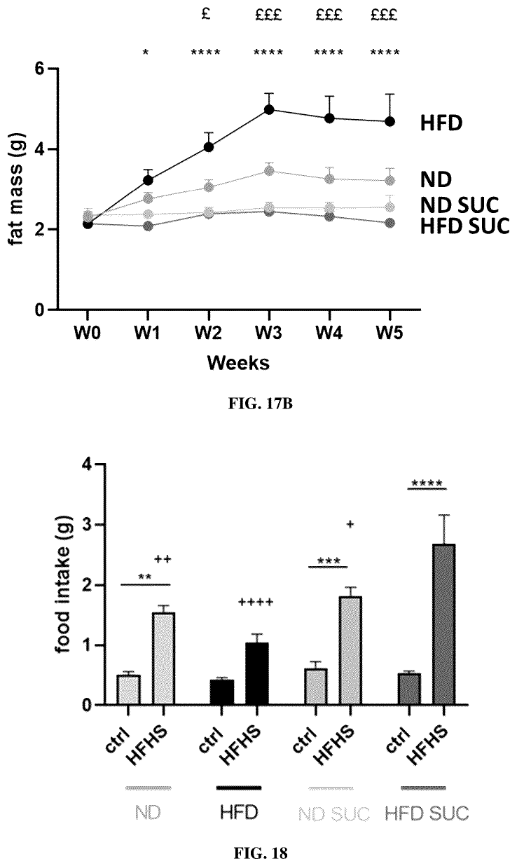

To assess effects of succinate on obese phenotype, mice were exposed to ND and HFD supplemented or not supplemented with sodium succinate at 5% w/w for eight weeks in ND SUCC/HFD SUCC and ND/HFD groups respectively (FIG. 17A-17B). As expected, mice fed a HFD showed a significant increase in body weight and fat mass compared to ND fed mice. In addition, mice fed a HFD diet combined with succinate also showed a significant decrease in body weight and fat mass compared to HFD mice. ND diet combined with succinate induced a significant decrease in body weight in ND SUCC mice compared to ND mice, however no significant change in fat mass was observed.

These results highlight potential beneficial effect of succinate supplementation in diet-induced obesity context.

### 2. Effects of Succinate on the Liking Component of Food Reward

As part of the study of hedonic food intake, a food preference test was performed at the fourth week of exposure to the different diets (ND and HFD). During this test, mice are exposed to a control diet (CT) as well as to a new food that is palatable (HFHS), thus allowing to assess the “liking” component of the food reward system (FIG. 18). When comparing the eaten amount of control (CT) and HFHS, mice in ND and ND SUCC groups eat significantly more HFHS than CT food (P<0.01; P<0.001). However, no significant difference was observed between the amount of HFHS ingested between ND and ND SUCC mice. For the CT and HFHS consumption by HFD mice, no significant difference was observed, in contrast to the HFD SUCC mice. HFD SUCC mice consumed more HFHS than CT in the test, but also more HFHS than mice in the ND, ND SUCC and HFD groups (P<0.01 ND HFHS vs. HFD SUCC HFHS; P<0.05 ND SUCC HFHS vs. HFD SUCC HFHS; P<0.0001 HFD HFHS vs. HFD SUCC HFHS).

These results highlight a potential involvement of succinate in the restoration of the liking component in the reward system associated with food.

This result is of particular interest in the context of the treatment of eating-related disorders. Indeed, the under-stimulation of the liking component of the reward system is known to lead to increased food consumption in order to attain a pleasurable stimulus; consequently, succinate may help in reducing food consumption in eating-related disorders (e.g., obesity-related disorders, binge eating and the like). The effects of succinate on the liking component may also be of interest for the treatment of other reward dysregulation disorders wherein the liking component is dysregulated.

### 3. Effects of Succinate on the Wanting Component of the Food Reward

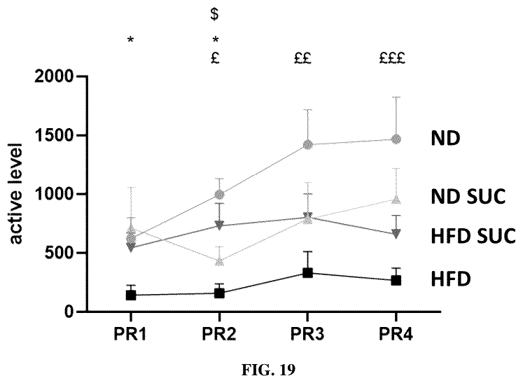

In order to further characterize the different components of the food reward system and in particular the motivation of the mice to obtain a food reward, (i.e., the “wanting” component of food intake), an operant wall test was performed and the motivation of the mice was assessed during the progressive ratio sessions (FIG. 19).

This test showed a significant decrease in the number of presses on the active lever to obtain sucrose pellets of HFD mice compared to ND mice during PR2 (P<0.05), PR3 (P<0.01) and PR4 sessions (P<0.001), reflecting a deficit in behaviour associated with the reward system component “wanting” in the context of obesity. A separate analysis among different progressive ratio sessions also indicates a significant increase in the number of active lever presses between HFD SUC and HFD mice during PR1 and PR2 and a significant decrease in the number of active lever presses of ND SUC mice compared to ND mice during PR2.

This test shows an effect of succinate on the wanting component of the reward system in obese and lean conditions.

These results support the use of succinate for treating eating-related disorders, and more generally reward for treating dysregulation disorders wherein the wanting component is over-stimulated, typically in patients having compulsive behavior towards a rewarding stimulus.

1-15. (canceled)

16. A method of preventing and/or treating reward dysregulation disorders; comprising administering, to an individual in need thereof, a composition comprising one or more bacteria from the genus Parabacteroides and/or an extract thereof and/or metabolites thereof.

17. The method according to claim 16, wherein the bacteria from the genus Parabacteroides are selected from the group consisting of P. distasonis, P. goldsteinii, P. merdae, P. acidifaciens, P. bouchesdurhonensis, P. chartae, P. chinchilla, P. chongii, P. faecis, P. gordonii, P. johnsonii, P. massiliensis, P. pacaensis, P. provencensis, P. timonensis, Parabacteroides spp. and combinations thereof.

18. The method according to claim 16, wherein the reward dysregulation disorder is selected in a group consisting of mental disorders, neurological disorders, and combinations thereof.

19. The method according to claim 16, wherein the reward dysregulation disorder is a mental disorder is selected from the group consisting of addiction-related disorder, eating-related disorder, affective disorders, obsessive compulsive disorders, schizophrenia, attention deficit hyperactivity disorders (ADHD), autism spectrum disorder, major depressive disorder (MDD), and anxiety disorder.

20. The method according to claim 16, wherein the reward dysregulation disorder is a mental disorder, said mental disorder being an eating-related disorder selected from the group consisting of anorexia, bulimia, binge eating, overweight-related disorders, and obesity-related disorders.

21. The method according to claim 16, wherein the reward dysregulation disorder is a mental disorder, said mental disorder being an addiction-related disorder selected from the group consisting of alcohol-related addiction, drug-related addiction, and game-related addiction.

22. The method according to claim 16, wherein the reward dysregulation disorder is a neurological disorder selected in a group consisting of Parkinson's disease, Tourette Syndrome.

23. The method according to claim 16, comprising administering the composition to a human individual.

24. The method according to claim 16, comprising administering the composition orally or rectally.

25. The method according to claim 16, comprising administering the bacteria at a dose comprised from about 1×10² CFU/g to about 1×10¹² CFU/g of the composition.

26. The method according to claim 16, wherein the composition further comprises one or more beneficial microbe(s).

27. The method according to claim 26, wherein the one or more beneficial microbe(s) is/are selected from group consisting of bacteria from the family Clostridiaceae, from the family Peptostreptococcaceae, from the family Prevotellaceae, from the family Methylobacteriaceae, from the genus Turicibacter, from the genus Coprococcus, from the genus Knoellia, from the genus Prevotella, from the genus Staphylococcus, and from the genus Akkermansiaceae.

28. The method according to claim 16, wherein the composition is in the form of a pharmaceutical composition further comprising a pharmaceutically acceptable carrier.

29. The method according to claim 16, wherein the composition is in the form of a nutritional composition further comprising a nutritionally acceptable carrier.

30. The method according to claim 16, wherein the composition is comprised in a kit, which further comprises means to administer said composition.

31. The method according to claim 16, wherein the reward dysregulation disorder is a mental disorder selected in a group consisting of anorexia and bulimia.

32. The method according to claim 16, wherein the reward dysregulation disorder is a mental disorder, said mental disorder being binge-eating.

33. The method according to claim 16, wherein the reward dysregulation disorder is a mental disorder, said mental disorder being an eating-related disorder that is not an overweight-related disorder or an obesity-related disorder.

34. A method of preventing and/or treating reward dysregulation disorders; comprising administering, to an individual in need thereof, a composition comprising succinate.

35. The method according to claim 34, wherein the succinate is produced by bacteria from the genus Parabacteroides.
